(19) United States
(12) Patent Application Publication
(10) Pub. No.: US 2024/0249408 A1
(43) Pub. Date: Jul. 25, 2024

(54) SYSTEMS AND METHODS FOR TIME OF FLIGHT IMAGING

(71) Applicant: Activ Surgical, Inc., Boston, MA (US)

(72) Inventors: Bogdan MITREA, Canton, MA (US); Nitish JAIN, Boston, MA (US); Adrian PARK, Boston, MA (US); Roman STOLYAROV, Cambridge, MA (US); Vasiliy BUHARIN, Winchester, MA (US); Michael VAL, Boston, MA (US); Charlie BEURSKENS, Boston, MA (US); Emanuel DEMAIO, Watertown, MA (US); Thomas CALEF, Bridgewater, MA (US); Suraj SRINIVASAN, Boston, MA (US); Peter KIM, Boston, MA (US)

(21) Appl. No.: 18/543,455

(22) Filed: Dec. 18, 2023

Related U.S. Application Data
(63) Continuation of application No. PCT/US22/34803, filed on Jun. 23, 2022.
(60) Provisional application No. 63/215,303, filed on Jun. 25, 2021.
(60) Provisional application No. 63/228,977, filed on Aug. 3, 2021.
(60) Provisional application No. 63/336,088, filed on Apr. 28, 2022.

(51) Int. Cl. G06T 7/00; G01S 17/894; G06T 7/593

(52) U.S. Cl. CPC G06T 7/0012; G01S 17/894; G06T 7/593; G06T 2207/10024; G06T 2207/10028; G06T 2207/10064; G06T 2207/10144; G06T 2207/20004; G06T 2207/30104

(57) ABSTRACT

The present disclosure provides a system and methods for time of flight imaging, comprising: (a) an imaging sensor configured to receive a plurality of light signals reflected from a surgical scene, wherein the imaging module comprises a first imaging unit configured for time of flight (TOF) imaging; a second imaging unit configured for at least one of laser speckle imaging and fluorescence imaging; and an optical element configured to (i) direct a first set of light signals to the first imaging unit and (ii) direct a second set of light signals to the second imaging unit; and (b) an image processing module operatively coupled to the first imaging unit and the second imaging unit, wherein the image processing module is configured to generate one or more images of the surgical scene based on the first set of light signals and the second set of light signals.

### CROSS-REFERENCE

This application is a continuation of International Patent Application No. PCT/US22/34803, filed on Jun. 23, 2022, which claims priority to U.S. Provisional Patent Application No. 63/215,303 filed on Jun. 25, 2021, U.S. Provisional Patent Application No. 63/228,977 filed on Aug. 3, 2021, and U.S. Provisional Patent Application No. 63/336,088 filed on Apr. 28, 2022, each of which is incorporated herein by reference in its entirety for all purposes.

### BACKGROUND

Medical imaging data may be used to capture images or videos associated with various anatomical, physiological, or morphological features within a medical or surgical scene.

### SUMMARY

The systems and methods disclosed herein may be used to generate accurate and useful imaging datasets that can be leveraged by medical or surgical operators to improve the precision, flexibility, and control of autonomous and/or semiautonomous robotic surgical systems. Such robotic surgical systems can further provide a medical or surgical operator with additional information, including, for example, live image overlays to enhance a medical operator's ability to perform one or more steps of a live surgical procedure quickly and efficiently in an optimal manner. Accurate laparoscopic three-dimensional (3D) profilometry can also enable various clinical applications including tissue measurement (such as tumors or hernias), distance correction in fluorescence imaging, and autonomous surgical robotics.

The systems and methods of the present disclosure may be implemented for medical imaging of a surgical scene using a variety of different imaging modalities. The medical images obtained or generated using the presently disclosed systems and methods may comprise, for example, time of flight (TOF) images, RGB images, depth maps, fluoroscopic images, laser speckle contrast images, hyperspectral images, multispectral images, or laser doppler images. The medical images may also comprise, for example, time of flight (TOF) videos, RGB videos, dynamic depth maps, fluoroscopic videos, laser speckle contrast videos, hyperspectral videos, multispectral videos, or laser doppler videos. In some cases, the medical imagery may comprise one or more streams of imaging data comprising a series of medical images obtained successively or sequentially over a time period. This method may, for example, compute depth values directly and/or generate a 3D point cloud of a target by estimating the travel time of optical pulses emitted by a laser source and captured by a synchronized camera and may be computationally inexpensive as it may use a pixel-wise distance calculation. The system includes an endoscopic TOF system that attains a dense, single-shot, sub-millimeter precision point cloud on a biological tissue target. The system may be capable of improving on the performance of the current known approaches by an order of magnitude in accuracy and three orders of magnitude in temporal resolution. The system employs near-infrared light and implemented using an endoscope, e.g., an off-the-shelf endoscope, suggesting integration ability with established imaging systems and minimal workflow interruption. These results may be attained on a 30 Hz acquisition system, suggesting feasibility of real-time application.

In some embodiments, the medical images may be processed to determine or detect one or more anatomical, physiological, or morphological processes or properties associated with the surgical scene or the subject undergoing a surgical procedure. As used herein, processing the medical images may comprise determining or classifying one or more features, patterns, or attributes of the medical images. In some embodiments, the medical images may be used to train or implement one or more medical algorithms or models for tissue tracking. In some embodiments, the systems and methods of the present disclosure may be used to augment various medical imagery with depth information.

In some embodiments, the one or more medical images may be used or processed to provide live guidance based on a detection of one or more tools, surgical phases, critical views, or one or more biological, anatomical, physiological, or morphological features in or near the surgical scene. In some embodiments, the one or more medical images may be used to enhance intra-operative decision making and provide supporting features (e.g., enhanced image processing capabilities or live data analytics) to assist a surgeon during a surgical procedure.

In some embodiments, the one or more medical images may be used to generate an overlay comprising (i) one or more RGB images or videos of the surgical scene and (ii) one or more additional images or videos of the surgical procedure, wherein the one or more additional images or videos comprise fluorescence data, laser speckle data, perfusion data, or depth information.

In an aspect, the present disclosure provides a system comprising: (a) an imaging module configured to receive a plurality of light signals reflected from a surgical scene, wherein the imaging module comprises: a first imaging unit configured for time of flight (TOF) imaging; a second imaging unit configured for at least one of laser speckle imaging and fluorescence imaging; and an optical element configured to (i) direct a first set of light signals to the first imaging unit and (ii) direct a second set of light signals to the second imaging unit; and (b) an image processing module operatively coupled to the first imaging unit and the second imaging unit, wherein the image processing module is configured to generate one or more images of the surgical scene based on the first set of light signals and the second set of light signals.

In some embodiments, the plurality of light signals comprises the first set of light signal and the second set of light signals. In some embodiments, the second imaging unit is configured for laser speckle imaging and fluorescence imaging. In some embodiments, the optical element comprises a beam splitter, a prism, or a mirror. In some embodiments, the mirror comprises a fast steering mirror or a dichroic mirror. In some embodiments, the prism comprises a trichroic prism assembly. In some embodiments, the optical element is configured to direct a third set of light signals to a third imaging unit configured for RGB imaging. The present disclosure relates to development of a neural network for full-field depth estimation of laparoscopic RGB surgical scenes. The presently disclosed methods attain clinical viability and enable supervision from a monocular laparoscopic depth camera.

In some embodiments, the system may further comprise a controller configured to control at least one of a gain, an exposure, a shutter timing, or a shutter size of at least one of the first imaging unit and the second imaging unit. In some embodiments, the controller is configured to control an exposure of the first imaging unit and the second imaging unit such that the first imaging unit receives the first set of light signals at a first point in time and the second imaging unit receives the second set of light signals at a second point in time, wherein the first point in time is different than the second point in time. In some embodiments, the controller is configured to control an exposure of the second imaging unit such that the second imaging unit receives a first subset of light signals for laser speckle imaging at a first point in time and a second subset of light signals for fluorescence imaging at a second point in time, wherein the first point in time is different than the second point in time. In some embodiments, the second set of light signals comprises the first subset of light signals and the second subset of light signals. In some embodiments, the second set of light signals received at the second imaging unit is generated using one or more time of flight light pulses transmitted to the surgical scene or a portion thereof. In some embodiments, the one or more time of flight light pulses are configured to excite one or more fluorescent particles or dyes in the surgical scene or cause the one or more fluorescent particles or dyes to fluoresce in order to produce the second set of light signals.

In some embodiments, the image processing module is configured to generate one or more images for visualizing fluorescence in the surgical scene, based on one or more light signals received at the first imaging unit. In some embodiments, the image processing module is configured to utilize image interpolation to account for a plurality of different frame rates and exposure times associated with the first and second imaging unit when generating the one or more images of the surgical scene. In some embodiments, the image processing module is configured to quantify or visualize perfusion of a biological fluid in, near, or through the surgical scene based on the one or more images of the surgical scene. In some embodiments, the image processing module is configured to generate one or more perfusion maps for one or more biological fluids in or near the surgical scene, based on the one or more images of the surgical scene. In some embodiments, the image processing module is configured to update, refine, or normalize the one or more perfusion maps based on a distance between (i) a scope through which the plurality of light signals can be transmitted and (ii) one or more pixels of the one or more images. In some embodiments, the image processing module is configured to update, refine, or normalize the one or more perfusion maps based on a position, an orientation, or a pose of a scope through which the plurality of light signals is transmitted relative to one or more pixels of the one or more images. In some embodiments, the image processing module is configured to update, refine, or normalize the one or more perfusion maps based on depth information or a depth map associated with the surgical scene, wherein the depth information or the depth map is derived from or generating using the first set of light signals. In some embodiments, the image processing module is configured to determine a pose of a scope through which the plurality of light signals can be transmitted relative to one or more pixels of the one or more images, based on the depth information or the depth map. In some embodiments, the image processing module is configured to update, refine, or normalize one or more velocity signals associated with the perfusion map based on the pose of the scope relative to the surgical scene. In some embodiments, the image processing module is configured to update, refine, or normalize the one or more perfusion maps based on at least one of (i) a type of tissue detected or identified within the surgical scene, (ii) an intensity of at least one of the first and second set of light signals, wherein the intensity is a function of a distance between a scope through which the plurality of light signals are transmitted and one or more pixels in the surgical scene, or (iii)on a spatial variation of an intensity of at least one of the first and second set of light signals across the surgical scene. In some embodiments, the image processing module is configured to infer a tissue type based on an intensity of one or more light signals reflected from the surgical scene, wherein the one or more reflected light signals comprise at least one of the first set of light signals and the second set of light signals.

In some embodiments, the system may further comprise a TOF light source configured to transmit the first set of light signals to the surgical scene. In some embodiments, the TOF light source is configured to generate and transmit one or more TOF light pulses to the surgical scene. In some embodiments, the TOF light source is configured to provide a spatially varying illumination to the surgical scene. In some embodiments, the TOF light source is configured to provide a temporally varying illumination to the surgical scene. In some embodiments, the TOF light source is configured to adjust an intensity of the first set of light signals. In some embodiments, the TOF light source is configured to adjust a timing at which the first set of light signals is transmitted. In some embodiments, the TOF light source is configured to adjust an amount of light directed to one or more regions in the surgical scene. In some embodiments, the TOF light source is configured to adjust one or more properties of the first set of light signals based on a type of surgical procedure, a type of tissue in the surgical scene, a type of scope through which the light signals are transmitted, or a length of a cable used to transmit the light signals from the TOF light source to a scope. In some embodiments, the one or more properties comprise a pulse width, a pulse repetition frequency, or an intensity.

In some embodiments, the image processing module is configured to use at least one of the first set of light signals and the second set of light signals to determine a motion of a scope, a tool, or an instrument relative to the surgical scene. In some embodiments, the image processing module is configured to (i) generate one or more depth maps or distance maps based on the first set of light signals or the second set of light signals, and (ii) use the one or more depth maps or distances map to generate one or more machine-leaming based inferences, which one or more machine-leaming based inferences comprise at least one of automatic video de-identification, image segmentation, automatic labeling of tissues or instruments in or near the surgical scene, and optimization of image data variability based on one or more normalized RGB or perfusion features. In some embodiments, the image processing module is configured to (i) generate one or more depth maps or distance maps based on at least one of the first set of light signals and the second set of light signals, and (ii) use the one or more depth maps or distances map to perform temporal tracking of perfusion or to implement speckle motion compensation. In some embodiments, the image processing module is operatively coupled to one or more 3D interfaces for viewing, assessing, or manipulating the one or more images, and the 3D interfaces comprise video goggles, a monitor, a light field display, or a projector.

In some embodiments, the system may further comprise a calibration module configured to perform depth calibration on one or more depth maps generated using the image processing module. In some embodiments, depth calibration comprises updating the one or more depth maps by sampling multiple targets at (i) multiple distances and/or (ii) multiple illumination intensities. In some embodiments, the system may further comprise a calibration module for calibrating (i) one or more light sources configured to provide the plurality of light signals or (ii) at least one of the first imaging unit and the second imaging unit. In some embodiments, the calibration module is configured to perform intrinsic calibration, which may comprise adjusting one or more intrinsic parameters associated with the first and/or second imaging units, wherein the one or more intrinsic parameters comprise a focal length, principal points, a distortion, or a field of view. In some embodiments, the calibration module is configured to perform acquisition parameter calibration, which may comprise adjusting one or more operational parameters associated with the first and/or second imaging units, wherein the one or more operational parameters comprise a shutter width, an exposure, a gain, or a shutter timing.

In some embodiments, the first set of light signals comprise one or more TOF light pulses with a wavelength of about 808 nanometers. In some embodiments, the second set of light signals comprise one or more laser speckle signals with a wavelength of about 852 nanometers. In some embodiments, the second set of light signals comprise one or more fluorescence signals with a wavelength ranging from about 800 nanometers to about 900 nanometers.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example imaging module for imaging a surgical scene using one or more imaging modalities, in accordance with some embodiments.

FIG. 2, FIG. 3, and FIG. 4 schematically illustrate various examples of different system configurations for implementing time of flight (TOF) imaging, in accordance with some embodiments.

FIG. 5 schematically illustrates an example method for TOF imaging, in accordance with some embodiments.

FIG. 6 schematically illustrates a TOF system calibration, measurement, and validation workflow, in accordance with some embodiments.

FIG. 7 schematically illustrates a TOF acquisition scheme in accordance with some embodiments.

FIG. 8 schematically illustrates a TOF depth calibration setup with 10 mm straight laparoscope.

FIG. 9 schematically illustrates a TOF accuracy evaluation setup with 10 mm straight laparoscope and 3D scanner.

FIG. 10A-10C schematically illustrate quantitative evaluation of 3D scanner on a 25 mm 3D-printed plastic hemisphere in accordance with some embodiments.

FIG. 11A-11E schematically illustrate depth map pre-processing steps as shown on images of a porcine kidney target in accordance with some embodiments.

FIG. 12 schematically illustrates mean nearest neighbor errors as a function of spatial and temporal filtering parameters. K refers to the spatial filter kernel size.

FIGS. 13A-13D schematically illustrate quantitative evaluation of endoscopic TOF point clouds of a porcine kidney using four sets of spatial and temporal filter orders in accordance with some embodiments.

FIG. 14 schematically illustrates examples of depth error distribution between time of flight (TOF) ground truth measurements and machine learning (ML) estimations or inferences.

FIG. 15 schematically illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### System

TOFImaging—In an aspect, the present disclosure provides systems and methods for time of flight (TOF) medical imaging. The terms “time of flight,” “time-of-flight,” “ToF,” or “TOF,” as used interchangeably herein, may generally refer to one or more measurements of a time taken by an object, a particle, or a wave to travel a distance through a medium (e.g., fluid, such as a liquid or gas). Examples of the wave may include acoustic wave and electromagnetic radiation. The time measurement(s) may be used to establish a velocity and/or a path length of the object, particle, or wave. In some cases, time of flight may refer to the time required for emitted electromagnetic radiation to travel from a source of the electronic radiation to a sensor (e.g., a camera). In some cases, a time of flight measurement may correspond to the time required for the emitted electromagnetic radiation to reach a target tissue. Alternatively, a time of flight measurement may correspond to the time required for the emitted electromagnetic radiation to reach a target tissue and to be directed or re-directed (e.g., reflected) to a sensor. Such sensor, which may comprise a TOF sensor, may be adjacent to the source of the emitted electromagnetic radiation, or may be at a different location than the source. In some cases, a camera or an imaging sensor may be used to determine a time of flight based on a phase shift of emitted and received signal (e.g., electromagnetic radiation). Examples of time of flight cameras may include, but are not limited to, radio frequency (RF)-modulated light sources with phase detectors (e.g., Photonic Mixer Devices (PMD), Swiss Ranger™, CanestaVision™), range gated imagers (e.g., ZCam™), and/or direct time-of-flight imagers (e.g., light detection and ranging (LIDAR)).

Imaging module—The present disclosure provides a system for performing or implementing TOF imaging. The system may comprise an imaging module configured to receive a plurality of light signals reflected from a surgical scene. The imaging module may comprise a first imaging unit configured for time of flight (TOF) imaging and a second imaging unit configured for at least one of laser speckle imaging and fluorescence imaging.

The first imaging unit and/or the second imaging unit may comprise one or more imaging devices. The imaging devices may comprise any imaging device configured to generate one or more medical images using light beams or light pulses transmitted to and reflected from a surgical scene. For example, the imaging devices may comprise a camera, a video camera, a three-dimensional (3D) depth camera, a stereo camera, a depth camera, a Red Green Blue Depth (RGB-D) camera, a time-of-flight (TOF) camera, an infrared camera, a charge coupled device (CCD) image sensor, and/or a complementary metal oxide semiconductor (CMOS) image sensor.

First Imaging Unit—The first imaging unit may comprise an imaging sensor configured for TOF imaging. The imaging sensor may be a TOF sensor. In some embodiments, the ToF sensor may utilize one or more aspects of heterodyne interferometry. The TOF sensor may be integrated with the first imaging unit. The TOF sensor may be configured to obtain one or more TOF light signals reflected from a surgical scene. The one or more TOF light signals may be used to generate a depth map of the surgical scene, based at least in part on a time it takes for light (e.g., a light wave, a light pulse, or a light beam) to travel from one or more portions of the surgical scene to a detector of the TOF sensor after being reflected off of the one or more portions of the surgical scene. In some cases, the one or more portions of the surgical scene may comprise, for example, one or more features that are present, visible, or detectable within the surgical scene. The one or more depth maps may be used to provide a medical operator with a more accurate real-time visualization of a depth of or a distance to a particular point or feature within the surgical scene. In some cases, the one or more depth maps may provide a surgeon with spatial information about the surgical scene to optimally maneuver a scope, robotic camera, robotic arm, or surgical tool relative to one or more features within the surgical scene.

TOF Sensor—The system may comprise a TOF sensor configured to receive at least a portion of the plurality of light beams or light pulses that are reflected from the surgical scene. The portion may comprise one or more TOF light beams or TOF light pulses reflected from the surgical scene. The TOF sensor may be configured to obtain one or more time of flight measurements associated with the reflected TOF light beams or TOF light pulses. In some cases, the time of flight measurements may correspond to the time required for emitted electromagnetic radiation to travel from a source of the electronic radiation to a sensor (e.g., the TOF sensor). In other cases, the time of flight measurements may correspond to the time required for the emitted electromagnetic radiation to reach a target tissue. Alternatively, the time of flight measurements may correspond to the time required for the emitted electromagnetic radiation to reach a target tissue and be directed (e.g., reflected back) to a TOF sensor.

In some cases, the TOF sensor may be positioned along a common beam path of the plurality of light beams or light pulses reflected from the surgical scene. The common beam path may be disposed between the surgical scene and an optical element that can be used to split the plurality of light beams or light pulses into different sets of light signals. In some cases, the plurality of light beams or light pulses reflected from the surgical scene may be split into (i) a first set of light signals corresponding to the TOF light and (ii) a second set of light signals corresponding to white light, laser speckle light, and/or fluorescence excitation light. The first set of light signals may have a beam path that is different than that of the second set of light signals and/or the plurality of light beams or light pulses reflected from the surgical scene. In such cases, the TOF sensor may be positioned along a discrete beam path of the first set of light signals that is downstream of the optical element.

In some cases, the TOF sensor may be positioned at a tip of a scope through which the plurality of light beams or light pulses are directed. In other cases, the TOF sensor may be attached to a portion of the surgical subject's body. The portion of the surgical subject's body may be proximal to the surgical scene being imaged or operated on.

In some embodiments, the system may comprise a plurality of depth sensing devices. Each of the plurality of depth sensing devices may be configured to obtain one or more TOF measurements used to generate a depth map of the surgical scene. The plurality of depth sensing devices may be selected from the group consisting of a stereo imaging device (e.g., a stereoscopic camera), a structured light imaging device, and a TOF depth sensor.

In some embodiments, the TOF sensor may comprise an imaging sensor configured to implement heterodyning to enable depth sensing and to enhance TOF resolution. Heterodyning can enable a slower sensor to sense depth, and may permit the use of regular camera sensors, instead of dedicated TOF hardware sensors, for TOF sensing.

Combined Sensor—In some cases, a single imaging sensor may be used for multiple types of imaging (e.g., TOF depth imaging and laser speckle imaging, TOF depth imaging and fluorescence imaging, laser speckle imaging and fluorescence imaging, or any combination of TOF depth imaging, laser speckle imaging, fluorescence imaging, and RGB imaging). In some cases, a single imaging sensor may be used for imaging based on multiple ranges of wavelengths, each of which may be specialized for a particular type of imaging or for imaging of a particular type of biological material or physiology.

In some cases, the TOF sensors described herein may comprise an imaging sensor configured for TOF imaging and at least one of RGB imaging, laser speckle imaging, and fluorescence imaging. In some cases, the imaging sensor may be configured for TOF imaging and perfusion imaging. In any of the embodiments described herein, the TOF sensor may be configured to see and register non-TOF light.

In some cases, the imaging sensor may be configured to capture TOF depth signals and laser speckle signals during alternating or different temporal slots. For example, the imaging sensor may capture a TOF depth signal at a first time instance, a laser speckle signal at a second time instance, a TOF depth signal at a third time instance, a laser speckle signal at a fourth time instance, and so on. The imaging sensor may be configured to capture a plurality of optical signals at different times. The optical signals may comprise a TOF depth signal, an RGB signal, a fluorescence signal, and/or a laser speckle signal.

In other cases, the imaging sensor may be configured to simultaneously capture TOF depth signals and laser speckle signals to generate one or more medical images comprising a plurality of spatial regions. The plurality of spatial regions may correspond to different imaging modalities. For example, a first spatial region of the one or more medical images may comprise a TOF depth image based on TOF measurements, and a second spatial region of the one or more medical images may comprise a laser speckle image based on laser speckle signals.

Second Imaging Unit—The second imaging unit may comprise an imaging sensor configured for at least one of laser speckle imaging and fluorescence imaging. In some embodiments, the second imaging unit may be configured for both laser speckle imaging and fluorescence imaging. The imaging sensor may comprise, for example, an imaging sensor for laser speckle imaging and/or a fluorescent light sensor. The laser speckle imaging sensor and/or the fluorescent light sensor may be configured to obtain one or more laser speckle or infrared light signals and/or one or more fluorescent light signals reflected from a surgical scene. The one or more laser speckle or infrared light signals and/or the one or more fluorescent light signals may be used to generate a laser speckle contrast image and/or a fluorescence image of one or more portions of the surgical scene. The one or more portions of the surgical scene may comprise, for example, one or more features that are present, visible, or detectable within the surgical scene.

Light Signals—As described above, the imaging module may be configured to receive a plurality of light signals reflected from a surgical scene. The plurality of light signals may comprise a first set of light signals and a second set of light signals. The first imaging unit may be configured to receive a first set of light signals reflected from the surgical scene. The second imaging unit may be configured to receive a second set of light signals reflected from the surgical scene. In some cases, an optical element (e.g., a mirror, a lens, a prism, a beam splitter, etc.) may be used to direct a first subset of the plurality of light signals to the first imaging unit and a second subset of the plurality of light signals to the second imaging unit. The first subset may correspond to the first set of light signals and the second subset may correspond to the second set of light signals. The first set of light signals may comprise one or more TOF light pulses with a wavelength of about 808 nanometers. The second set of light signals may comprise one or more laser speckle signals with a wavelength of about 852 nanometers. In some cases, the second set of light signals may comprise one or more fluorescence signals with a wavelength ranging from about 800 nanometers to about 900 nanometers.

Optical Element—In some embodiments, the imaging module may comprise an optical element. The optical element may be configured to (i) direct a first set of light signals to the first imaging unit and (ii) direct a second set of light signals to the second imaging unit. In some cases, the optical element may comprise a beam splitter, a prism, or a mirror. In some cases, the prism may comprise a trichroic prism assembly. In some cases, the mirror may comprise a fast steering mirror or a dichroic mirror.

In some cases, the optical element may be configured to direct a third set of light signals to a third imaging unit configured for RGB imaging. The third imaging unit may comprise a camera or an imaging sensor for RGB imaging of the surgical scene. In some cases, the third imaging unit may be releasably coupled to the imaging module. In some cases, the third imaging unit may comprise a third party camera. In some cases, the third imaging unit may be integrated with the imaging module.

Time Sharing—In some embodiments, the system may further comprise a controller configured to control at least one of a gain, an exposure, a shutter timing, or a shutter size of at least one of the first imaging unit and the second imaging unit. In some cases, the controller may be configured to control an exposure of the first imaging unit and the second imaging unit such that the first imaging unit receives the first set of light signals at a first point in time and the second imaging unit receives the second set of light signals at a second point in time. This may be referred to as time sharing among different sensors. The first set of light signals may comprise TOF light, which may be used by the first imaging unit for TOF imaging. The second set of light signals may comprise laser speckle light and/or fluorescent light, which may be used by the second imaging unit for laser speckle imaging and/or fluorescence imaging. The first point in time may be different than the second point in time.

In some cases, the controller may be configured to control an exposure of the second imaging unit such that the second imaging unit receives a first subset of light signals for laser speckle imaging at a first point in time and a second subset of light signals for fluorescence imaging at a second point in time. This may be referred to as time sharing for a same sensor. The second set of light signals received at the second imaging unit may comprise the first subset of light signals and the second subset of light signals as described herein. The first subset of light signals may comprise laser speckle light, which may be used by the second imaging unit for laser speckle imaging. The second subset of light signals may comprise fluorescent light, which may be used by the second imaging unit for fluorescence imaging. The fluorescent light may be associated with one or more dyes (e.g., ICG dyes) or autofluorescence of one or more biological materials (e.g., organs, tissue, biological fluids such as blood, etc.). The first point in time may be different than the second point in time.

Light Sources—In some embodiments, the system may comprise a plurality of light sources. The plurality of light sources may comprise a time of flight (TOF) light source configured to generate TOF light. In some cases, the plurality of light sources may further comprise at least one of a white light source, a laser speckle light source, and a fluorescence excitation light source. In other cases, the plurality of light sources may not or need not comprise a white light source, a laser light source, or a fluorescence excitation light source.

TOFLight Source—The TOF light source may comprise a laser or a light emitting diode (LED). The laser or the light emitting diode (LED) may be configured to generate a TOF light. The TOF light may comprise an infrared or near infrared light having a wavelength from about 700 nanometers (nm) to about 1 millimeter (mm). In some cases, the TOF light may comprise visible light having a wavelength from about 400 nm to about 700 nm. In some cases, the visible light may comprise blue light having a wavelength from about 400 nm to about 500 nm. Advantages of visible light for TOF applications include low penetration of tissue surfaces, which can improve the reliability and accuracy of TOF measurements. In contrast, IR light, which penetrates tissue surfaces deeper and induces multi-reflections, can (a) cause ambiguity as to which internal surface or subsurface is reflecting the IR light, and (b) introduce errors in any TOF measurements obtained. In some cases, the TOF light may comprise a plurality of light beams and/or light pulses having a plurality of wavelengths from about 400 nm to about 1 mm.

In some embodiments, the TOF light source may be used to generate a plurality of TOF light pulses. In such cases, the TOF light source may be pulsed (i.e., switched ON and OFF at one or more predetermined intervals). In some cases, such pulsing may be synced to an opening and/or a closing of one or more TOF camera shutters.

In some embodiments, the TOF light source may be used to generate a continuous TOF light beam. In some cases, the TOF light source may be continuously ON, and a property of the TOF light may be modulated. For example, the continuous TOF light beam may undergo an amplitude modulation. The amplitude modulated TOF light beam may be used to obtain one or more TOF measurements based on a phase difference between the emitted TOF light and the reflected TOF light. The TOF depth measurements may be computed based at least in part on a phase shift observed between the TOF light directed to the target region and the TOF light reflected from the target region. In other cases, when the TOF light source is used to generate a continuous TOF light beam, one or more movable mechanisms (e.g., an optical chopper or a physical shuttering mechanism such as an electromechanical shutter or gate) may be used to generate a series of TOF pulses from the continuous TOF light beam. The plurality of TOF light pulses may be generated by using a movement of the electromechanical shutter or gate to chop, split, or discretize the continuous light beam into the plurality of TOF light pulses. The advantage of having the TOF light beam continuously on is that there are no delays in ramp-up and ramp-down (i.e., no delays associated with powering the beam on and off).

In some embodiments, the TOF light source may be located remote from a scope and operatively coupled to the scope via a light guide. For example, the TOF light source may be located on or attached to a surgical tower. In other embodiments, the TOF light source may be located on the scope and configured to provide the TOF light to the scope via a scope-integrated light guide. The scope-integrated light guide may comprise a light guide that is attached to or integrated with a structural component of the scope. The light guide may comprise a thin filament of a transparent material, such as glass or plastic, which is capable of transmitting light signals through successive internal reflections. Alternatively, the TOF light source may be configured to provide the TOF light to the target region via one or more secondary illuminating scopes. In such cases, the system may comprise a primary scope that is configured receive and direct light generated by other light sources (e.g., a white light source, a laser speckle light source, and/or a fluorescence excitation light source). The one or more secondary illuminating scopes may be different than the primary scope. The one or more secondary illuminating scopes may comprise a scope that is separately controllable or movable by a medical operator or a robotic surgical system. The one or more secondary illuminating scopes may be provided in a first set of positions or orientations that is different than a second set of positions or orientations in which the primary scope is provided. In some cases, the TOF light source may be located at a tip of the scope. In other cases, the TOF light source may be attached to a portion of the surgical subject's body. The portion of the surgical subject's body may be proximal to the target region being imaged using the medical imaging systems of the present disclosure. In any of the embodiments described herein, the TOF light source may be configured to illuminate the target region through a rod lens. The rod lens may comprise a cylindrical lens configured to enable beam collimation, focusing, and/or imaging. In some cases, the TOF light source may be configured to illuminate the target region through a series or a combination of lenses (e.g., a series of relay lenses).

In some embodiments, the system may comprise a TOF light source configured to transmit the first set of light signals to the surgical scene. The TOF light source may be configured to generate and transmit one or more TOF light pulses to the surgical scene. In some cases, the TOF light source may be configured to provide a spatially varying illumination to the surgical scene. In some cases, the TOF light source may be configured to provide a temporally varying illumination to the surgical scene.

In any of the embodiments described herein, the timing of the opening and/or closing of one or more shutters associated with the one or more imaging units may be adjusted based on the spatial and/or temporal variation of the illumination. In some cases, the image acquisition parameters for the one or more imaging units may be tuned based on the surgical application (e.g., type of surgical procedure), a scope type, or a cable length. In some cases, the TOF acquisition scheme may be tuned based on a distance between the surgical scene and one or more components of the TOF imaging systems disclosed herein.

In some cases, the TOF light source may be configured to adjust an intensity of the first set of light signals. In some cases, the TOF light source may be configured to adjust a timing at which the first set of light signals is transmitted. In some cases, the TOF light source may be configured to adjust an amount of light directed to one or more regions in the surgical scene. In some cases, the TOF light source may be configured to adjust one or more properties of the first set of light signals based on a type of surgical procedure, a type of tissue in the surgical scene, a type of scope through which the light signals are transmitted, or a length of a cable used to transmit the light signals from the TOF light source to a scope. The one or more properties may comprise, for example, a pulse width, a pulse repetition frequency, or an intensity.

In some cases, the TOF light source may be configured to generate a plurality of light pulses, light beams, or light waves for TOF imaging. In some cases, the TOF light source may be configured to generate light pulses, light beams, or light waves having multiple different wavelengths or ranges of wavelengths.

ICG excitation with TOF light pulses—In some cases, the TOF light source may be configured to generate one or more light pulses, light beams, or light waves with a wavelength of about 808 nanometers, about 825 nanometers, or about 792 nanometers. In some cases, the TOF light source may be configured to generate one or more light pulses, light beams, or light waves that are usable for TOF imaging and/or fluorescence imaging.

In some embodiments, the second set of light signals received at the second imaging unit may be generated using one or more time of flight light pulses transmitted to and/or reflected from the surgical scene or a portion thereof. In some cases, the one or more time of flight light pulses may be configured to excite one or more fluorescent particles or dyes in the surgical scene or cause the one or more fluorescent particles or dyes to fluoresce in order to produce the second set of light signals. The second set of light signals may comprise one or more fluorescent light signals associated with dye fluorescence or tissue autofluorescence. In some cases, one or more pulsed TOF signals may be used to excite one or more dyes (e.g., ICG dyes) in or near the surgical scene.

In some cases, the system may comprise an image processing module. The image processing module may be configured for visualization of ICG fluorescence based on one or more reflected light signals associated with one or more light pulses generated using a TOF light source. The image processing module may be configured for visualization of ICG fluorescence based on one or more signals or measurements obtained using a TOF sensor. In some cases, the image processing module may be configured to generate one or more images for visualizing fluorescence in the surgical scene, based on one or more light signals received at the first imaging unit.

TOFApplications—In some embodiments, the TOF measurements obtained using the systems and methods of the present disclosure may be used to learn, detect, and/or monitor the movements of a human operator or a surgical tool, or to track human three-dimensional (3D) kinematics. In some cases, the TOF measurements may be used to generate one or more depth or distance maps for machine-leaming based inferences, including, for example, automatic video de-identification and/or tool or tissue segmentation. In some cases, the TOF measurements may be used to normalize RGB or prefusion features to decrease data variability, which can help to identify, for example, bile ducts in a critical view of safety. In some cases, the TOF measurements may be used to generate distance or depth maps for automatic labeling of tools or tissues within the surgical scene. In some cases, the TOF measurements may be used to perform temporal tracking of perfusion characteristics or other features within the surgical scene. In some cases, the TOF measurements may be used for speckle motion compensation.

In some cases, the measurements and/or the light signals obtained using one or more imaging sensors of the imaging module may be used for perfusion quantification. In some cases, the measurements and/or the light signals obtained using one or more imaging sensors may be used to generate, update, and/or refine one or more perfusion maps for the surgical scene. In some cases, the perfusion maps may be refined or adjusted based on a pixelwise distance or depth compensation using one or more TOF depth measurements. In other cases, the perfusion maps may be refined or adjusted based on a global distance or depth compensation using one or more TOF depth measurements. In some cases, the perfusion maps may be updated or calibrated based on one or more baseline or reference distances and depths. In some cases, the TOF measurements may be used to generate a distance map, which may be used to estimate a pose of one or more instruments (e.g., a scope) in or near the surgical scene. In some cases, the pose estimate may be used to compensate one or more velocity signals associated with a movement of a tool or an instrument or a movement of a biological material (e.g., blood) in or near the surgical scene.

White Light Source—The white light source may be configured to generate one or more light beams or light pulses having one or more wavelengths that lie within the visible spectrum. The white light source may comprise a lamp (e.g., an incandescent lamp, a fluorescent lamp, a compact fluorescent lamp, a halogen lamp, a metal halide lamp, a fluorescent tube, a neon lamp, a high intensity discharge lamp, or a low pressure sodium lamp), a light bulb (e.g., an incandescent light bulb, a fluorescent light bulb, a compact fluorescent light bulb, or a halogen light bulb), and/or a light emitting diode (LED). The white light source may be configured to generate a white light beam. The white light beam may be a polychromatic emission of light comprising one or more wavelengths of visible light. The one or more wavelengths of light may correspond to a visible spectrum of light. The one or more wavelengths of light may have a wavelength between about 400 nanometers (nm) and about 700 nanometers (nm). In some cases, the white light beam may be used to generate an RGB image of a target region.

Laser Speckle Light Source—The laser speckle light source may comprise one or more laser light sources. The laser speckle light source may comprise one or more light emitting diodes (LEDs) or laser light sources configured to generate one or more laser light beams with a wavelength between about 700 nanometers (nm) and about 1 millimeter (mm). In some cases, the one or more laser light sources may comprise two or more laser light sources that are configured to generate two or more laser light beams having different wavelengths. The two or more laser light beams may have a wavelength between about 700 nanometers (nm) and about 1 millimeter (mm). The laser speckle light source may comprise an infrared (IR) laser, a near-infrared laser, a short-wavelength infrared laser, a mid-wavelength infrared laser, a long-wavelength infrared laser, and/or a far-infrared laser. The laser speckle light source may be configured to generate one or more light beams or light pulses having one or more wavelengths that lie within the invisible spectrum. The laser speckle light source may be used for laser speckle imaging of a target region.

Fluorescence Excitation Light Source—In some cases, the plurality of light sources may comprise a fluorescence excitation light source. The fluorescence excitation light source may be used for fluorescence imaging. As used herein, fluorescence imaging may refer to the imaging of any fluorescent materials (e.g., auto fluorescing biological materials such as tissues or organs) or fluorescing materials (e.g., dyes comprising a fluorescent substance like fluorescein, coumarin, cyanine, rhodamine, or any chemical analog or derivative thereof). The fluorescence excitation light source may be configured to generate a fluorescence excitation light beam. The fluorescence excitation light beam may cause a fluorescent dye (e.g., indocyanine green) to fluoresce (i.e., emit light). The fluorescence excitation light beam may have a wavelength of between about 600 nanometers (nm) and about 900 nanometers (nm). The fluorescence excitation light beam may be emitted onto a target region. In some cases, the target region may comprise one or more fluorescent dyes configured to absorb the fluorescence excitation light beam and re-emit fluorescent light with a wavelength between about 750 nanometers (nm) and 950 nanometers (nm). In some cases, the one or more fluorescent dyes may be configured to absorb the fluorescence excitation light beam and to re-emit fluorescent light with a wavelength that ranges from about 700 nanometers to about 2.5 micrometers (μm).

In some cases, the fluorescence excitation light source may be configured to generate one or more light pulses, light beams, or light waves with a wavelength of about 808 nanometers, about 825 nanometers, or about 792 nanometers. In some cases, the fluorescence excitation light source may be configured to generate one or more light pulses, light beams, or light waves that are usable for fluorescence imaging and/or TOF imaging.

Beams Pulses—In any of the embodiments described herein, the plurality of light sources may be configured to generate one or more light beams. In such cases, the plurality of light sources may be configured to operate as a continuous wave light source. A continuous wave light source may be a light source that is configured to produce a continuous, uninterrupted beam of light with a stable output power.

In some cases, the plurality of light sources may be configured to continuously emit pulses of light and/or energy at predetermined intervals. In such cases, the light sources may be switched on for limited time intervals and may alternate between a first power state and a second power state. The first power state may be a low power state or an OFF state. The second power state may be a high power state or an ON state.

Alternatively, the plurality of light sources may be operated in a continuous wave mode, and the one or more light beams generated by the plurality of light sources may be chopped (i.e., separated, or discretized) into a plurality of light pulses using a mechanical component (e.g., a physical object) that blocks the transmission of light at predetermined intervals. The mechanical component may comprise a movable plate that is configured to obstruct an optical path of one or more light beams generated by the plurality of light sources, at one or more predetermined time periods.

TOF Light Modulator—In some embodiments, the system may further comprise a TOF light modulator. The TOF light modulator may be configured to adjust one or more properties (e.g., illumination intensity, direction of propagation, travel path, etc.) of the TOF light generated using the TOF light source. In some cases, the TOF light modulator may comprise a diverging lens that is positioned along a light path of the TOF light. The diverging lens may be configured to modulate an illumination intensity of the TOF light across the target region. In other cases, the TOF light modulator may comprise a light diffusing element that is positioned along a light path of the TOF light. The light diffusing element may likewise be configured to modulate an illumination intensity of the TOF light across the target region. Alternatively, the TOF light modulator may comprise a beam steering element configured to illuminate the target region and one or regions proximal to the target region. The beam steering element may be used to illuminate a greater proportion of a scene comprising the target region. In some cases, the beam steering element may comprise a lens or a mirror (e.g., a fast steering mirror).

TOFParameter Optimizer—In some embodiments, the system may further comprise a TOF parameter optimizer configured to adjust one or more pulse parameters and one or more camera parameters, based at least in part on the application, depth range, tissue type, scope type, or procedure type. The one or more TOF measurements obtained using the TOF sensor may be based at least in part on the one or more pulse parameters and the one or more camera parameters. For example, the TOF parameter optimizer may be used to implement a first set of pulse parameters and camera parameters for a first procedure, and to implement a second set of pulse parameters and camera parameters for a second procedure. In some cases, the first procedure and the second procedure may have different depth ranges of interest. The TOF parameter optimizer may be configured to adjust the one or more pulse parameters and/or the one or more camera parameters to improve a resolution, accuracy, or tolerance of TOF depth sensing, and to increase the TOF signal to noise ratio for TOF applications. In some cases, the TOF parameter optimizer may be configured to determine the actual or expected performance characteristics of the TOF depth sensing system based on a selection or adjustment of one or more pulse parameters or camera parameters. Alternatively, the TOF parameter optimizer may be configured to determine a set of pulse parameters and camera parameters required to achieve a resolution, accuracy, or tolerance for a depth range or surgical operation.

In some cases, the TOF parameter optimizer may be configured to adjust the one or more pulse parameters and the one or more camera parameters in real time. In other cases, the TOF parameter optimizer may be configured to adjust the one or more pulse parameters and the one or more camera parameters offline. In some cases, the TOF parameter optimizer may be configured to adjust the one or more pulse parameters and/or camera parameters based on a feedback loop. The feedback loop may be implemented using a controller (e.g., a programmable logic controller, a proportional controller, a proportional integral controller, a proportional derivative controller, a proportional integral derivative controller, or a fuzzy logic controller). In some cases, the feedback loop may comprise a real-time control loop that is configured to adjust the one or more pulse parameters and/or the one or more camera parameters based on a temperature of the TOF light source or the TOF sensor. In some embodiments, the system may comprise an image post processing unit configured to update the depth map based on an updated set of TOF measurements obtained using the one or more adjusted pulse parameters or camera parameters.

The TOF parameter optimizer may be configured to adjust one or more pulse parameters. The one or more pulse parameters may comprise, for example, an illumination intensity, a pulse width, a pulse shape, a pulse count, a pulse on/off level, a pulse duty cycle, a TOF light pulse wavelength, a light pulse rise time, and a light pulse fall time. The illumination intensity may correspond to an amount of laser power used to provide a sufficient detectable TOF light signal during a laparoscopic procedure. The pulse width may correspond to a duration of the pulses. The TOF system may require a time of flight laser pulse of some minimal or maximal duration to guarantee a certain acceptable depth resolution. The pulse shape may correspond to a phase, an amplitude, or a period of the pulses. The pulse count may correspond to a number of pulses provided within a predetermined time period. Each of the pulses may have at least a predetermined amount of power (in Watts) in order to enable single pulse time of flight measurements with reduced noise. The pulse on/off level may correspond to a pulse duty cycle. The pulse duty cycle may be a function of the ratio of pulse duration or pulse width (PW) to the total period (T) of the pulse waveform. The TOF pulse wavelength may correspond to a wavelength of the TOF light from which the TOF light pulse is derived. The TOF pulse wavelength may be predetermined or adjusted accordingly for each TOF application. The pulse rise time may correspond to an amount of time for the amplitude of a pulse to rise to a selected or predetermined peak pulse amplitude. The pulse fall time may correspond to an amount of time for the peak pulse amplitude to fall to a selected or predetermined value. The pulse rise time and/or the pulse fall time may be modulated to meet a certain threshold value. In some cases, the TOF light source may be pulsed from a lower power mode (e.g., 50%) to higher power mode (e.g., 90%) to minimize rise time. In some cases, a movable plate may be used to chop a continuous TOF light beam into a plurality of TOF light pulses, which can also minimize or reduce pulse rise time.

The TOF parameter optimizer may be configured to adjust one or more camera parameters. The camera parameters may include, for example, a number of shutters, shutter timing, shutter overlap, shutter spacing, and shutter duration. As used herein, a shutter may refer to a physical shutter and/or an electronic shutter. A physical shutter may comprise a movement of a shuttering mechanism (e.g., a leaf shutter or a focal-plane shutter of an imaging device or imaging sensor) in order to control exposure of light to the imaging device or imaging sensor. An electronic shutter may comprise turning one or more pixels of an imaging device or imaging sensor ON and/or OFF to control exposure. The number of shutters may correspond to a number of times in a predetermined time period during which the TOF camera is shuttered open to receive TOF light pulses. In some cases, two or more shutters may be used for a TOF light pulse. Temporally spaced shutters can be used to deduce the depth of features in the target region. In some cases, a first shutter may be used for a first pulse (e.g., an outgoing pulse), and a second shutter may be used for a second pulse (e.g., an incoming pulse). Shutter timing may correspond to a timing of shutter opening and/or shutter closing based on a timing of when a pulse is transmitted and/or received. The opening and/or closing of the shutters may be adjusted to capture one or more TOF pulses or a portion thereof. In some cases, the shutter timing may be adjusted based on a path length of the TOF pulses or a depth range of interest. Shutter timing modulation may be implemented to minimize the duty cycle of TOF light source pulsing and/or camera shutter opening and closing, which can enhance the operating conditions of the TOF light source and improve HW longevity (e.g., by limiting or controlling the operating temperature). Shutter overlap may correspond to a temporal overlap of two or more shutters. Shutter overlap may increase peak Rx power at short pulse widths where peak power is not immediately attained. Shutter spacing may correspond to the temporal spacing or time gaps between two or more shutters. Shutter spacing may be adjusted to time the TOF camera shutters to receive the beginning and/or the end of the pulse. Shutter spacing may be optimized to increase the accuracy of TOF measurements at decreased Rx power. Shutter duration may correspond to a length of time during which the TOF camera is shuttered open to receive TOF light pulses. Shutter duration may be modulated to minimize noise associated with a received TOF light signal, and to ensure that the TOF camera receives a minimum amount of light used for TOF depth sensing applications.

In some cases, hardware may be interchanged or adjusted in addition to or in lieu of software-based changes to pulse parameters and camera parameters, in order to achieve the depth sensing capabilities for a particular depth sensing application.

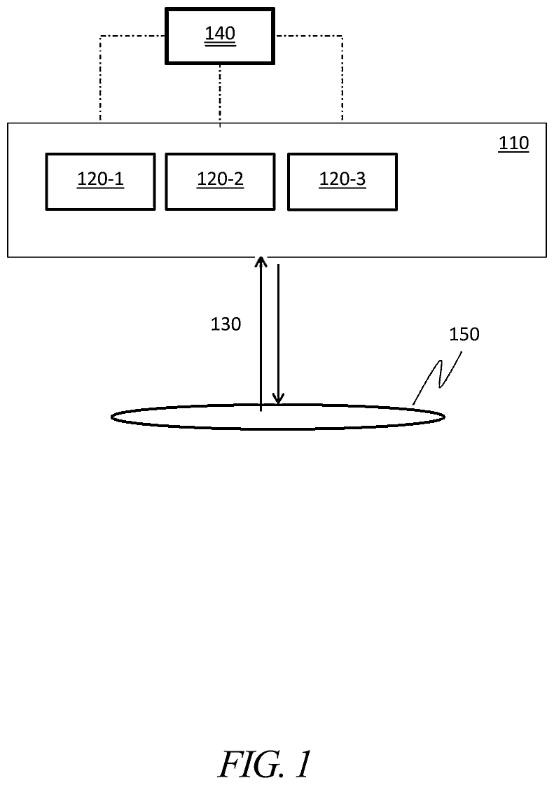

FIG. 1 schematically illustrates an example of an imaging module 110 for time of flight (TOF) imaging. The imaging module 110 may comprise a plurality of imaging units 120-1, 120-2, 120-3, etc. The plurality of imaging units 120-1, 120-2, 120-3 may comprise one or more imaging sensors. The imaging sensors may be configured for different types of imaging (e.g., TOF imaging, RGB imaging, laser speckle imaging, and/or fluorescence imaging). In some cases, the plurality of imaging units 120-1, 120-2, 120-3 may be integrated into the imaging module 110. In other cases, at least one of the plurality of imaging units 120-1, 120-2, 120-3 may be provided separately from the imaging module 110.

The imaging module may be configured to receive one or more signals reflected from a surgical scene 150. The one or more signals reflected from a surgical scene 150 may comprise one or more optical signals. The one or more optical signals may correspond to one or more light waves, light pulses, or light beams generated using a plurality of light sources. The plurality of light sources may comprise one or more light sources for TOF imaging, RGB imaging, laser speckle imaging, and/or fluorescence imaging. The one or more optical signals may be generated when the one or more light waves, light pulses, or light beams generated using the plurality of light sources are transmitted to and reflected from the surgical scene 150. In some cases, the one or more light waves, light pulses, or light beams generated using the plurality of light sources may be transmitted to the surgical scene 150 via a scope (e.g., a laparoscope). In some cases, the reflected optical signals from the surgical scene 150 may be transmitted back to the imaging module 110 via the scope. The reflected optical signals (or a subset thereof) may be directed to the appropriate imaging sensor and/or the appropriate imaging units 120-1, 120-2, 120-3.

The imaging units 120-1, 120-2, 120-3 may be operatively coupled to an image processing module 140. As described in greater detail below, the image processing module 140 may be configured to generate one or more images of the surgical scene 150 based on the optical signals received at the imaging units 120-1, 120-2, 120-3. In some cases, the image processing module 140 may be provided separately from the imaging module 110. In other cases, the image processing module 140 may be integrated with or provided as a component within the imaging module 110.

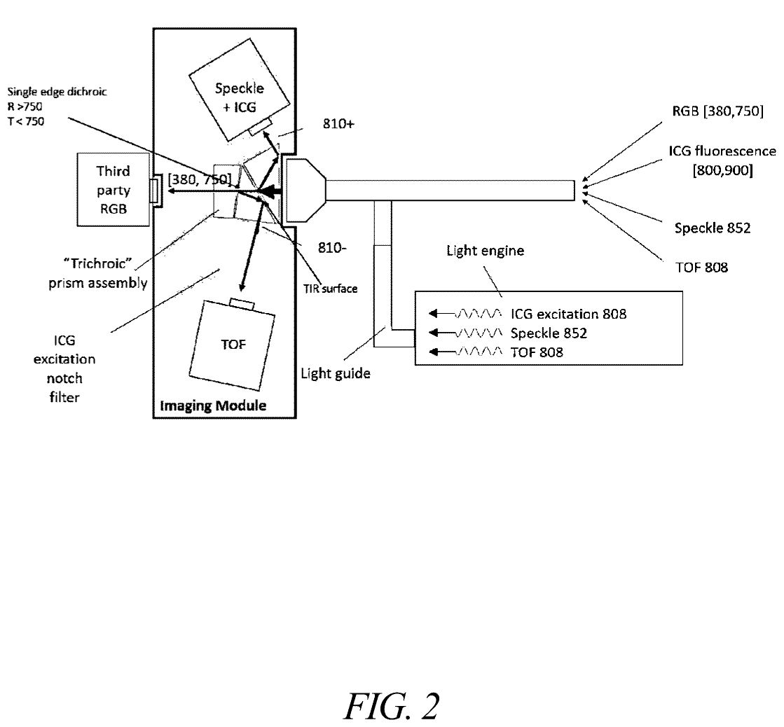

FIG. 2, FIG. 3, and FIG. 4 illustrate various other examples of a TOF imaging system. The TOF imaging system may comprise an imaging module. The imaging module may be operatively coupled to a scope. The scope may be configured to receive one or more input light signals from one or more light sources. The one or more input light signals may be transmitted from the one or more light sources to the scope via a light guide. The one or more input light signals may comprise, for example, white light for RGB imaging, fluorescence excitation light for fluorescence imaging, infrared light for laser speckle imaging, and/or time of flight (TOF) light for TOF imaging. The one or more input light signals may be transmitted through a portion of the scope and directed to a target region (e.g., a surgical scene). At least a portion of light signals transmitted to the target region may be reflected back through the scope to the imaging module. The imaging module may be configured to receive the reflected light signals, and to direct different subsets or portions of the reflected light signals to one or more imaging units to enable various types of imaging based on different imaging modalities. In some cases, the imaging module may comprise one or more optical elements for splitting the reflected light signals into the different subsets of light signals. Such splitting may occur based on a wavelength of the light signals, or a range of wavelengths associated with the light signals. The optical elements may comprise, for example, a mirror, a lens, or a prism. The optical element may comprise a dichroic mirror, a trichroic mirror, a dichroic lens, a trichroic lens, a dichroic prism, and/or a trichroic prism. In some cases, the optical elements may be placed adjacent to each other.

As shown in FIG. 2, in some cases the input light signals generated by the plurality of light sources may comprise ICG excitation light having a wavelength that ranges from about 800 nanometers to about 900 nanometers, laser speckle light having a wavelength that ranges from about 800 nanometers to about 900 nanometers, and TOF light having a wavelength that ranges from about 800 nanometers to about 900 nanometers. In some cases, the ICG excitation light may have a wavelength of about 808 nanometers. In some cases, the laser speckle light may have a wavelength of about 852 nanometers. In some cases, the TOF light may have a wavelength of about 808 nanometers.

The light signals reflected from the target region or surgical scene may be directed through the scope to one or more optical elements in the imaging module. In some cases, the one or more optical elements may be configured to direct a first subset of the reflected light signals to a first imaging unit for TOF imaging. In some cases, the one or more optical elements may be configured to direct a second subset of the reflected light signals to a second imaging unit for laser speckle imaging and/or fluorescence imaging. The first and second subsets of the reflected light signals may be separated based on a threshold wavelength. The threshold wavelength may be, for example, about 810 nanometers. In some cases, the one or more optical elements may be configured to permit a third subset of the reflected light signals to pass through to a third imaging unit. The third imaging unit may comprise a camera for RGB imaging. The third imaging unit may be a third party imaging unit that may be coupled to the imaging module. In some embodiments, the imaging module may comprise a notch filter for ICG excitation light.

FIG. 3 illustrates another example of a TOF imaging system. The TOF imaging system illustrated in FIG. 3 may be similar to the TOF imaging system of FIG. 2 and may not or need not require a notch filter for the ICG excitation light. The TOF imaging system shown in FIG. 3 may be configured to receive one or more reflected light signals that are generated using one or more input light signals provided by a plurality of light sources. The one or more input light signals may comprise ICG excitation light having a wavelength that ranges from about 800 nanometers to about 900 nanometers, laser speckle light having a wavelength that ranges from about 800 nanometers to about 900 nanometers, and TOF light having a wavelength that ranges from about 800 nanometers to about 900 nanometers. In some cases, the ICG excitation light may have a wavelength of about 825 nanometers. In some cases, the laser speckle light may have a wavelength of about 852 nanometers. In some cases, the TOF light may have a wavelength of about 808 nanometers.

FIG. 4 illustrates another example of a TOF imaging system. The TOF imaging system illustrated in FIG. 4 may be similar to the TOF imaging system of FIG. 2 and FIG. 3. In some cases, the TOF imaging system may comprise an ICG excitation notch filter. In other cases, the TOF imaging system may not or need not require a notch filter for the ICG excitation light. The TOF imaging system shown in FIG. 4 may be configured to receive one or more reflected light signals that are generated using one or more input light signals provided by a plurality of light sources. The one or more input light signals may comprise laser speckle light having a wavelength that ranges from about 800 nanometers to about 900 nanometers, and a set of light signals for both TOF imaging and ICG excitation. The set of light signals for both TOF imaging and ICG excitation may have a wavelength that ranges from about 800 nanometers to about 900 nanometers. In some cases, the laser speckle light may have a wavelength of about 852 nanometers. In some cases, the set of light signals for both TOF imaging and ICG excitation may have a wavelength of about 808 nanometers

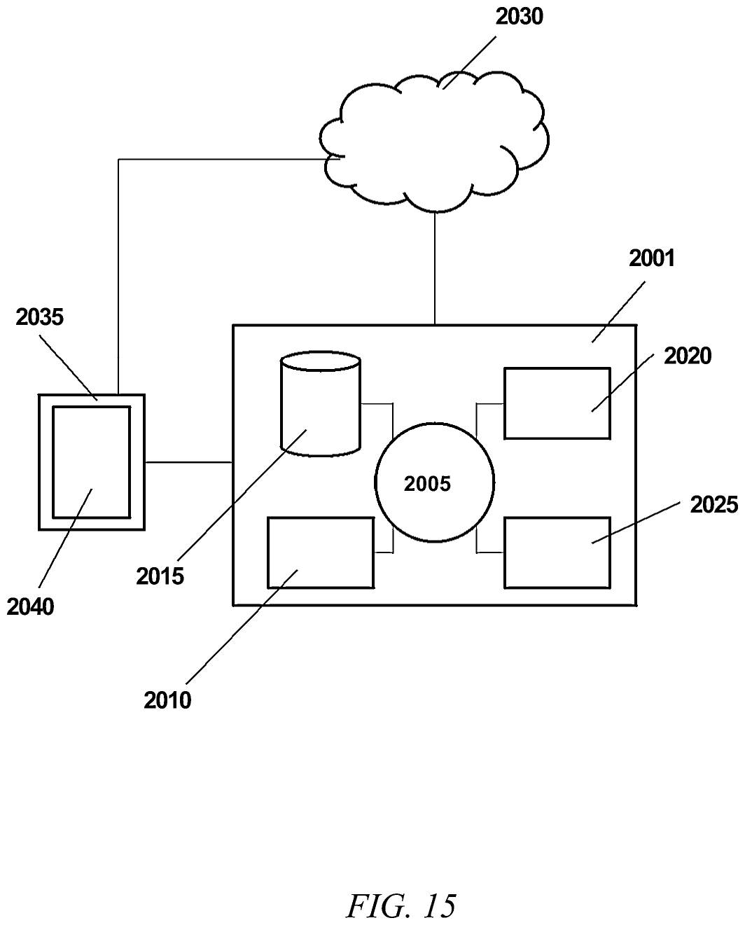

TOF Camera—Hardware: The TOF method described herein may employ a 3D camera development platform. The 3D camera development platform may use a 12-bit VGA CCD sensor to capture a depth map and corresponding intensity map at 30 Hz. The kit may comprise a camera board. The camera board may comprise on or more of the following: a CCD, CCD signal processor, and various peripherals. The kit may comprise an illumination board. The illumination board may comprise four independently controlled 940 nm VCSEL driver circuits. Each or either of these boards may also report a local temperature measurement. The camera board may interface with a host processing board. The host processing board may run Linux Debian. The host processing board may render frames directly or relay them to a laptop. In some case, the host processing board may comprise an example, variation, or embodiment of computer processor 2001, as described elsewhere herein with respect to FIG. 15.

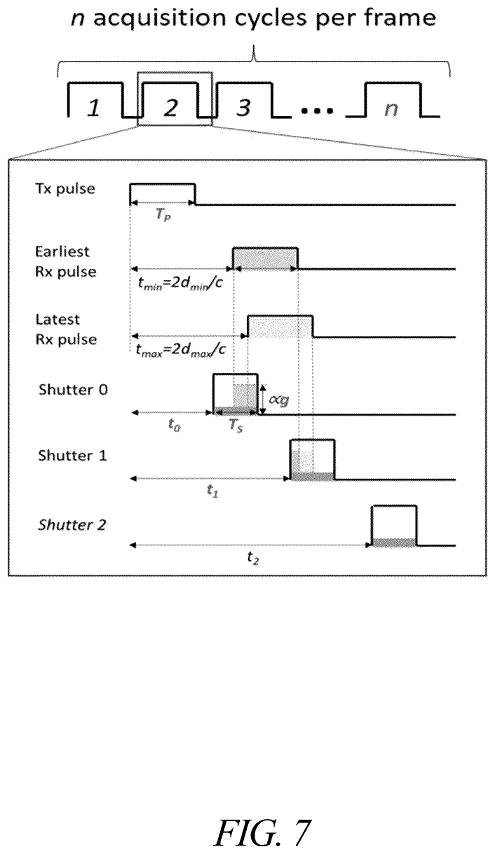

TOF Camera—Acquisition system: The acquisition system may employ a TOF measurement unit. A TOF measurement unit may comprise a triple-gated pulsed TOF measurement (visualized in FIG. 7), in which signal shutters (e.g. two) are used to capture the rising and falling edges of returned laser pulses, and a noise shutter or shutters is used to sample ambient light. The depth estimate \hat{d} at a given pixel is based on the computation

$$\hat{d} = af ( \frac{S 0 - S 2}{S 0 + S 1 - 2 * S 2} ) + b ,$$

where S₀, S₁, and S₂ are respectively the total integrated signals from the first signal shutter, second signal shutter, and noise shutter, a and b are pixel-wise constants, and f( ) is a non-linear correction function. As many as 90000 pulses may be emitted during a single frame, 45000 for each signal shutter. Synchronization between laser pulses and shutters is maintained by a closed feedback loop on pulse timing.

Laser subsystem: In some cases, systems and methods disclosed herein may employ an illumination board. An illumination board may comprise a plurality of vertical cavity surface-emitting lasers (VCSELs). An illumination board may comprise driver circuits for a plurality of VCSELs. In some cases, the plurality of VCSELs comprises four diffused 940 nm. In some cases, the plurality of VSCELS is intended for free-space application. In some cases, a endoscope, such as an off-the-shelf endoscope for surgical applications, may have significant scattering and absorption losses. In order to increase the received optical signal power at the CCD, the illumination electronics may be modified to increase optical power output. In some examples, modifications which increase optical power output may comprise one or more of: incorporating 850 nm VCSELs as opposed to the native 940 nm VCSELs due to improved CCD quantum efficiency and better transmission properties of most endoscopes at wavelengths closer to the visible range; incorporating a 60 degree diffuser into each VCSEL package as opposed to the native 110 degree diffuser; incorporating a 0.15Ω series resistor in each laser driver circuit as opposed to the native 0.33Ω resistor; and powering VCSELs at 6V as opposed to their native 2.5V supply to increase optical power.

Mechanical Assembly—Optical transmit path: In some cases, light emitted from a single VCSEL is coupled into a multimode fiber bundle that is then fed directly to a 10 mm 0-degree laparoscope. The fiber bundle may be fixed on both ends with mechanical adapters to prevent motion.

Mechanical Assembly—Optical receive path: In some cases, a laparoscope may be attached to a camera head coupler. To enable a focused image, a coupler may be mechanically attached to the camera board S-mount via CS-mount-to-C-mount spacers and S-mount-to-C-mount adapter. In some cases, in order to diminish optical noise, an 850 nm bandpass filter with a full width half maximum of 10 nm may be incorporated or placed directly in front of the CCD.

Cooling system: Two 30 mm 12V brushless DC fans may be positioned directly below, oriented toward, and sufficiently close to the laser driver circuits on the modified laser board. In some cases, the fans may be askew a few degrees to create flow along the board surface. The fans may stabilize the reported illumination board temperature during extended VCSEL operation.

### Methods

Image Processing Module—In some embodiments, the system may further comprise an image processing module (e.g., image processing module 140) operatively coupled to the first imaging unit and the second imaging unit. The image processing module may be configured to generate one or more images of the surgical scene based on the first set of light signals and the second set of light signals.

In some cases, the image processing module may be configured to utilize image interpolation to account for a plurality of different frame rates and exposure times associated with the first and second imaging unit when generating the one or more images of the surgical scene. In some cases, the image processing module may be configured to quantify or visualize perfusion of a biological fluid in, near, or through the surgical scene based on the one or more images of the surgical scene. In some cases, the image processing module may be configured to generate one or more perfusion maps for one or more biological fluids in or near the surgical scene, based on the one or more images of the surgical scene. In some cases, the image processing module may be configured to update, refine, or normalize the one or more perfusion maps based on a distance between (i) a scope through which the plurality of light signals is transmitted and (ii) one or more pixels of the one or more images. In some cases, the image processing module may be configured to update, refine, or normalize the one or more perfusion maps based on a position, an orientation, or a pose of a scope through which the plurality of light signals is transmitted relative to one or more pixels of the one or more images. In some cases, the image processing module may be configured to update, refine, or normalize the one or more perfusion maps based on depth information or a depth map associated with the surgical scene. The depth information or the depth map may be derived from or generating using the first set of light signals. In some cases, the image processing module may be configured to determine a pose of a scope through which the plurality of light signals is transmitted relative to one or more pixels of the one or more images, based on the depth information or the depth map. In some cases, the image processing module may be configured to update, refine, or normalize one or more velocity signals associated with the perfusion map based on the pose of the scope relative to the surgical scene. In some cases, the image processing module may be configured to update, refine, or normalize the one or more perfusion maps based on a type of tissue detected or identified within the surgical scene. In some cases, the image processing module may be configured to update, refine, or normalize the one or more perfusion maps based on an intensity of at least one of the first and second set of light signals. The intensity of the light signals may be a function of a distance between a scope through which the plurality of light signals is transmitted and one or more pixels in the surgical scene. In some cases, the image processing module may be configured to update, refine, or normalize the one or more perfusion maps based on a spatial variation of an intensity of at least one of the first and second set of light signals across the surgical scene. In some cases, the image processing module may be configured to infer a tissue type based on an intensity of one or more light signals reflected from the surgical scene, wherein the one or more reflected light signals comprise at least one of the first set of light signals and the second set of light signals. In some cases, the image processing module may be configured to use at least one of the first set of light signals and the second set of light signals to determine a motion of a scope, a tool, or an instrument relative to the surgical scene.

In some cases, the image processing module may be configured to (i) generate one or more depth maps or distance maps based on the first set of light signals or the second set of light signals, and (ii) use the one or more depth maps or distances map to generate one or more machine-leaming based inferences. The one or more machine-learning based inferences may comprise at least one of automatic video de-identification, image segmentation, automatic labeling of tissues or instruments in or near the surgical scene, and optimization of image data variability based on one or more normalized RGB or perfusion features. In some cases, the image processing module may be configured to (i) generate one or more depth maps or distance maps based on at least one of the first set of light signals and the second set of light signals, and (ii) use the one or more depth maps or distances map to perform temporal tracking of perfusion or to implement speckle motion compensation.

In some cases, the image processing module may be operatively coupled to one or more 3D interfaces for viewing, assessing, or manipulating the one or more images. For example, the image processing module may be configured to provide the one or more images to the one or more 3D interfaces for viewing, assessing, or manipulating the one or more images. In some cases, the one or more 3D interfaces may comprise video goggles, a monitor, a light field display, or a projector.

In some cases, the image processing module may be configured to generate a depth map of the surgical scene based at least in part on one or more TOF measurements obtained using the TOF sensor. The image processing module may comprise any of the imaging devices or imaging sensors described herein. In some cases, the image processing module may be integrated with one or more imaging devices or imaging sensors. The depth map may comprise an image or an image channel that contains information relating to a distance or a depth of one or more surfaces or regions within the surgical scene, relative to a reference viewpoint. The reference viewpoint may correspond to a location of a TOF depth sensor relative to one or more portions of the surgical scene. The depth map may comprise depth values for a plurality of points or locations within the surgical scene. The depth values may correspond to a distance between (i) a TOF depth sensor or a TOF imaging device and (ii) a plurality of points or locations within the surgical scene.

Overlay—In some cases, the image processing module may be configured to generate one or more image overlays comprising the one or images generated using the image processing module. The one or more image overlays may comprise a superposition of at least a portion of a first image on at least a portion of a second image. The first image and the second image may be associated with different imaging modalities (e.g., TOF imaging, laser speckle imaging, fluorescence imaging, RGB imaging, etc.). The first image and the second image may correspond to a same or similar region or set of features of the surgical scene. Alternatively, the first image and the second image may correspond to different regions or sets of features of the surgical scene. The one or more images generated using the image processing module may comprise the first image and the second image.

In some cases, the image processing module may be configured to provide or generate an overlay of a perfusion map and a live image of a surgical scene. In some cases, the image processing module may be configured to provide or generate an overlay of a perfusion map and a pre-operative image of a surgical scene. In some cases, the image processing module may be configured to provide or generate an overlay of a pre-operative image of a surgical scene and a live image of the surgical scene, or an overlay of a live image of the surgical scene with a pre-operative image of the surgical scene. The overlay may be provided in real time as the live image of the surgical scene is being obtained during a live surgical procedure. In some cases, the overlay may comprise two or more live images or videos of the surgical scene. The two or more live images or videos may be obtained or captured using different imaging modalities (e.g., TOF imaging, RGB imaging, fluorescence imaging, laser speckle imaging, etc.).

In some cases, the image processing module may be configured to provide augmented visualization by way of image or video overlays, or additional video data corresponding to different imaging modalities. An operator using the TOF imaging systems and methods disclosed herein may select various types of imaging modalities or video overlays for viewing. In some examples, the imaging modalities may comprise, for example, RGB imaging, laser speckle imaging, time of flight depth imaging, ICG fluorescence imaging, tissue autofluorescence imaging, or any other type of imaging using a predetermined range of wavelengths. The video overlays may comprise, in some cases, perfusion views and/or ICG fluorescence views. Such video overlays may be performed in real-time. The overlays may be performed live when a user toggles the overlay using one or more physical or graphical controls (e.g., buttons or toggles). The various types of imaging modalities and the corresponding visual overlays may be toggled on and off by the user (e.g., by clicking a button or a toggle). In some cases, the image processing module may be configured to provide or generate a first processed image or video corresponding to a first imaging modality (TOF) and a second processed video corresponding to a second imaging modality (laser speckle, fluorescence, RGB, etc.). The user may view the first processed video for a first portion of the surgical procedure, and switch or toggle to the second processed video for a second portion of the surgical procedure. Alternatively, the user may view an overlay comprising the first processed video and the second processed video, wherein the first and second processed video correspond to a same or similar time frame during which one or more steps of a surgical procedure are being performed.

In some cases, the image processing module may be configured to process or pre-process medical imaging data (e.g., surgical images or surgical videos) in real-time as the medical imaging data is being captured.

TOF Calibration—In some embodiments, the system may further comprise a calibration module configured to perform depth calibration on one or more depth maps generated using the image processing module. In some cases, depth calibration may comprise updating the one or more depth maps by sampling multiple targets at (i) multiple distances and/or (ii) multiple illumination intensities. In some cases, the system may further comprise a calibration module for calibrating (i) one or more light sources configured to provide the plurality of light signals or (ii) at least one of the first imaging unit and the second imaging unit.

In some cases, the calibration module may be configured to perform intrinsic calibration. Intrinsic calibration may comprise adjusting one or more intrinsic parameters associated with the first and/or second imaging units. The one or more intrinsic parameters may comprise, for example, a focal length, principal points, a distortion, and/or a field of view.

In some cases, the calibration module may be configured to perform acquisition parameter calibration. Acquisition parameter calibration may comprise adjusting one or more operational parameters associated with the first and/or second imaging units. The one or more operational parameters may comprise, for example, a shutter width, an exposure, a gain, and/or a shutter timing.

Normalizing Images—In some embodiments, the system may further comprise an image post processing unit configured to normalize an RGB image of the target region, a fluorescent image of the target region, or speckle based flow and perfusion signals associated with the target region, based at least in part on one or more TOF depth measurements obtained using the TOF sensor.

In some cases, an image of the target region may exhibit shading effects that are not visually representative of the actual target region. When an image of a surgical scene is obtained by illuminating the surgical scene with light directed through a scope (e.g., a laparoscope, an endoscope, a borescope, a videoscope, or a fiberscope), the image may comprise a radial shading gradient. The radial shading gradient may correspond to a light intensity fall-off pattern that varies as a function of an inverse square of a distance from a center point of illumination. The light intensity fall-off pattern may also vary as a function of a distance from a tip of the scope to the center point of illumination. The light intensity fall-off pattern may be a function of (i) a vertical distance from a tip of the scope to a center point of illumination within the surgical scene and (ii) a horizontal distance from the center point of illumination to the one or more pixels of the initial image. The one or more TOF depth measurements obtained using the TOF sensor may be used to reduce or eliminate misleading, deceiving, or erroneous shading effects present within an image generated using RGB data, laser speckle signals, and/or fluorescence characteristics.

In some cases, the image post processing unit may be configured to use an illumination profile of the target region and a distance between the scope and the target region being imaged to correct for image intensity at a periphery of one or more RGB or fluorescent images obtained using light pulses or light beams transmitted through the scope. In some cases, the image post processing unit may be configured to use a constant hematocrit concentration (i.e., the proportion of blood that comprises red blood cells, by volume) to estimate blood flow velocity through or proximal to the target region.

Surgical Procedures—The systems and methods of the present disclosure may be implemented to perform TOF imaging for various types of surgical procedures. The surgical procedure may comprise one or more general surgical procedures, neurosurgical procedures, orthopedic procedures, and/or spinal procedures. In some cases, the one or more surgical procedures may comprise colectomy, cholecystectomy, appendectomy, hysterectomy, thyroidectomy, and/or gastrectomy. In some cases, the one or more surgical procedures may comprise hernia repair, and/or one or more suturing operations. In some cases, the one or more surgical procedures may comprise bariatric surgery, large or small intestine surgery, colon surgery, hemorrhoid surgery, and/or biopsy (e.g., liver biopsy, breast biopsy, tumor, or cancer biopsy, etc.).

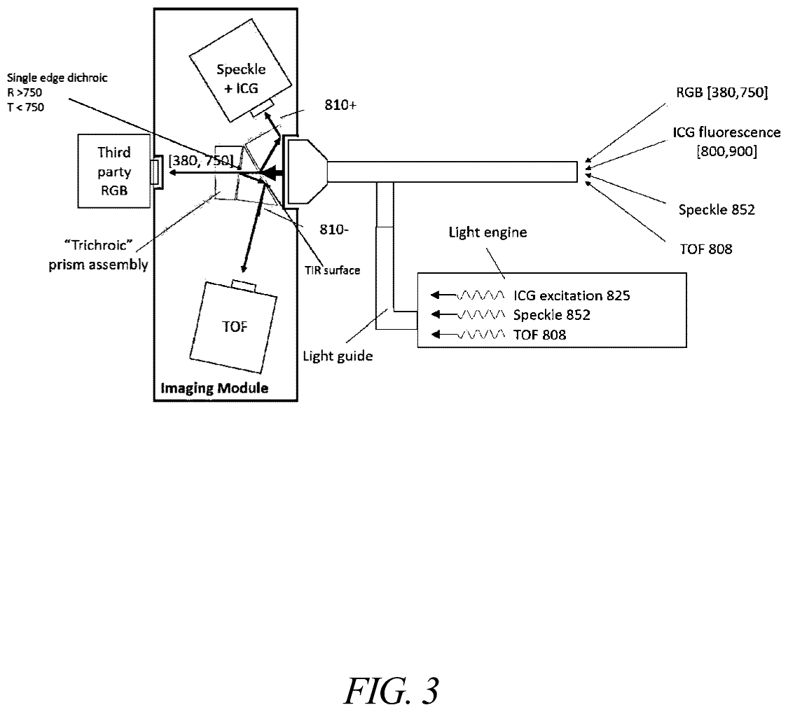

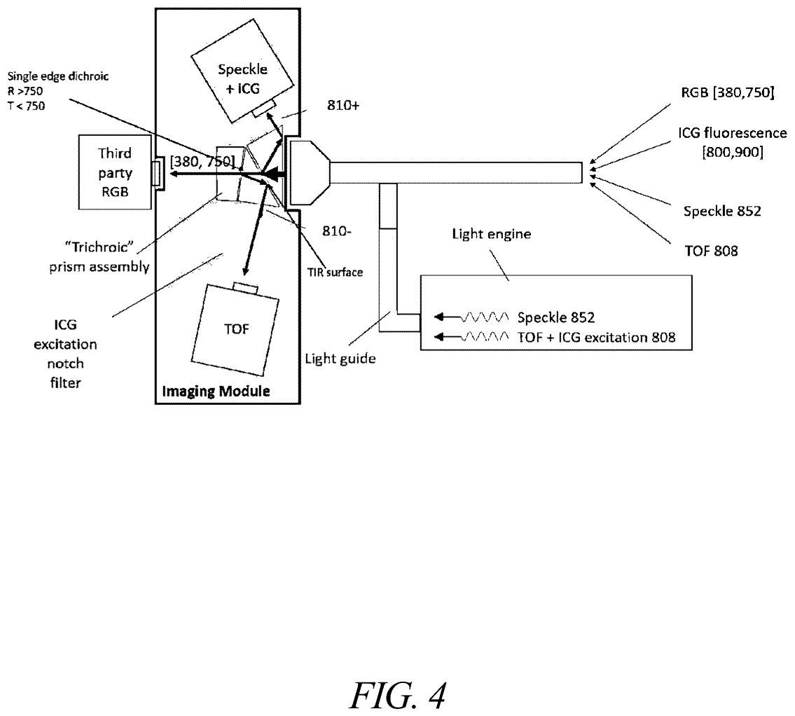

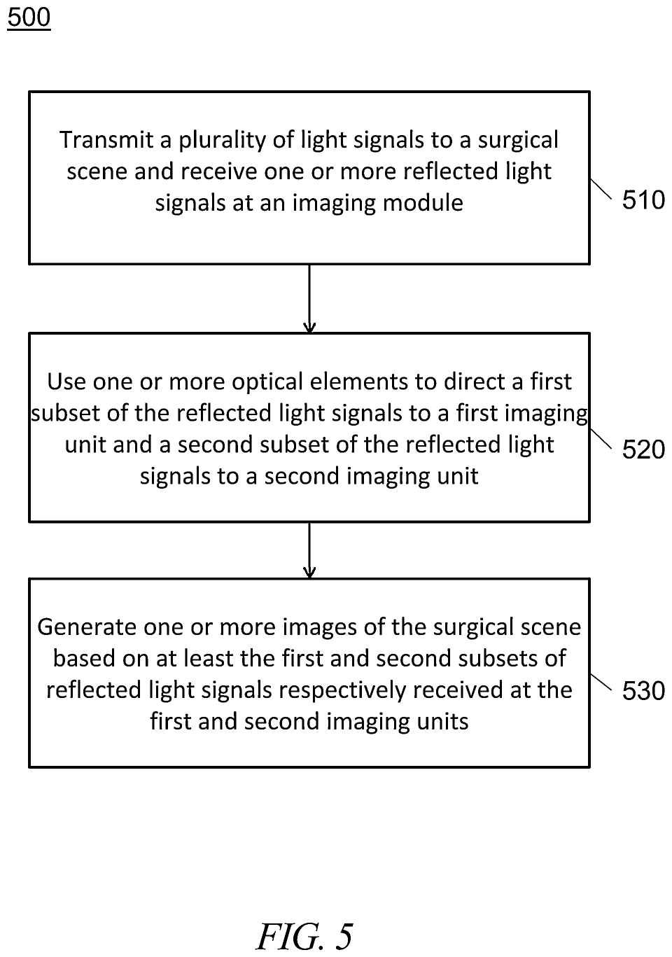

FIG. 5 illustrates an example method 500 for time of flight imaging. The method may comprise a step 510 comprising (a) transmitting a plurality of light signals to a surgical scene and receiving one or more reflected light signals from the surgical scene at an imaging module. The method may comprise another step 520 comprising (b) using one or more optical elements to direct a first subset of the reflected light signals to a first imaging unit and a second subset of the reflected light signals to a second imaging unit. The method may comprise another step 530 comprising (c) generating one or more images of the surgical scene based on at least the first and second subsets of reflected light signals respectively received at the first and second imaging units. The first subset of reflected light signals may be used for TOF imaging. The second subset of reflected light signals may be used for laser speckle imaging and/or fluorescence imaging.

### Computer Systems

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein. In another aspect, the present disclosure provides computer systems that are programmed or otherwise configured to implement methods of the disclosure. Referring to FIG. 15, the computer system 2001 may be programmed or otherwise configured to implement a method for TOF imaging. The computer system can be an electronic device of a user or a computer system that is remotely located with respect to the electronic device. The electronic device can be a mobile electronic device.

The computer system 2001 may be configured to, for example, control a transmission of a plurality of light signals to a surgical scene. The plurality of light signals may be reflected from the surgical scene, and the one or more reflected light signals from the surgical scene may be received at an imaging module. One or more optical elements of the imaging module may be used to direct a first subset of the reflected light signals to a first imaging unit and a second subset of the reflected light signals to a second imaging unit. The system may be further configured to generate one or more images of the surgical scene based on at least the first and second subsets of reflected light signals respectively received at the first and second imaging units. The first subset of reflected light signals may be used for TOF imaging. The second subset of reflected light signals may be used for laser speckle imaging and/or fluorescence imaging. The computer system 2001 can be an electronic device of a user or a computer system that is remotely located with respect to the electronic device. The electronic device can be a mobile electronic device. In some cases, computer system 2001 comprises a example, variation, or embodiment of image processing module 140 as described herein with respect to FIG. 1.

The computer system 2001 may include a central processing unit (CPU, also “processor” and “computer processor” herein) 2005, which can be a single core or multi core processor, or a plurality of processors for parallel processing. The computer system 2001 also includes memory or memory location 2010 (e.g., random-access memory, read-only memory, flash memory), electronic storage unit 2015 (e.g., hard disk), communication interface 2020 (e.g., network adapter) for communicating with one or more other systems, and peripheral devices 2025, such as cache, other memory, data storage and/or electronic display adapters. The memory 2010, storage unit 2015, interface 2020 and peripheral devices 2025 are in communication with the CPU 2005 through a communication bus (solid lines), such as a moth-erboard. The storage unit 2015 can be a data storage unit (or data repository) for storing data. The computer system 2001 can be operatively coupled to a computer network (“network”) 2030 with the aid of the communication interface 2020. The network 2030 can be the Internet, an internet and/or extranet, or an intranet and/or extranet that is in communication with the Internet. The network 2030 in some cases is a telecommunication and/or data network. The network 2030 can include one or more computer servers, which can enable distributed computing, such as cloud computing. The network 2030, in some cases with the aid of the computer system 2001, can implement a peer-to-peer network, which may enable devices coupled to the computer system 2001 to behave as a client or a server.

The CPU 2005 can execute a sequence of machine-readable instructions, which can be embodied in a program or software. The instructions may be stored in a memory location, such as the memory 2010. The instructions can be directed to the CPU 2005, which can subsequently program or otherwise configure the CPU 2005 to implement methods of the present disclosure. Examples of operations performed by the CPU 2005 can include fetch, decode, execute, and writeback.

The CPU 2005 can be part of a circuit, such as an integrated circuit. One or more other components of the system 2001 can be included in the circuit. In some cases, the circuit is an application specific integrated circuit (ASIC).

The storage unit 2015 can store files, such as drivers, libraries, and saved programs. The storage unit 2015 can store user data, e.g., user preferences and user programs. The computer system 2001 in some cases can include one or more additional data storage units that are located external to the computer system 2001 (e.g., on a remote server that is in communication with the computer system 2001 through an intranet or the Internet).

The computer system 2001 can communicate with one or more remote computer systems through the network 2030. For instance, the computer system 2001 can communicate with a remote computer system of a user (e.g., a doctor, a surgeon, an operator, a healthcare provider, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2001 via the network 2030.

Methods as described herein can be implemented by way of machine (e.g., computer processor) executable code stored on an electronic storage location of the computer system 2001, such as, for example, on the memory 2010 or electronic storage unit 2015. The machine executable or machine readable code can be provided in the form of software. During use, the code can be executed by the processor 2005. In some cases, the code can be retrieved from the storage unit 2015 and stored on the memory 2010 for ready access by the processor 2005. In some situations, the electronic storage unit 2015 can be precluded, and machine-executable instructions are stored on memory 2010.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 2001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2001 can include or be in communication with an electronic display 2035 that comprises a user interface (UI) 2040 for providing, for example, a portal for a doctor or a surgeon to view one or more medical images associated with a live procedure. The portal may be provided through an application programming interface (API). A user or entity can also interact with various elements in the portal via the UI. Examples of UI's include, without limitation, a graphical user interface (GUI) and web-based user interface.

### EXAMPLES

The examples and embodiments described herein are for illustrative purposes only and are not intended to limit the scope of the claimed invention. Various modifications or changes in light of the examples and embodiments described herein will be suggested to persons skilled in the art and are to be include within the spirit and purview of this application and scope of the appended claims.

### Calibration and Measurement Workflow

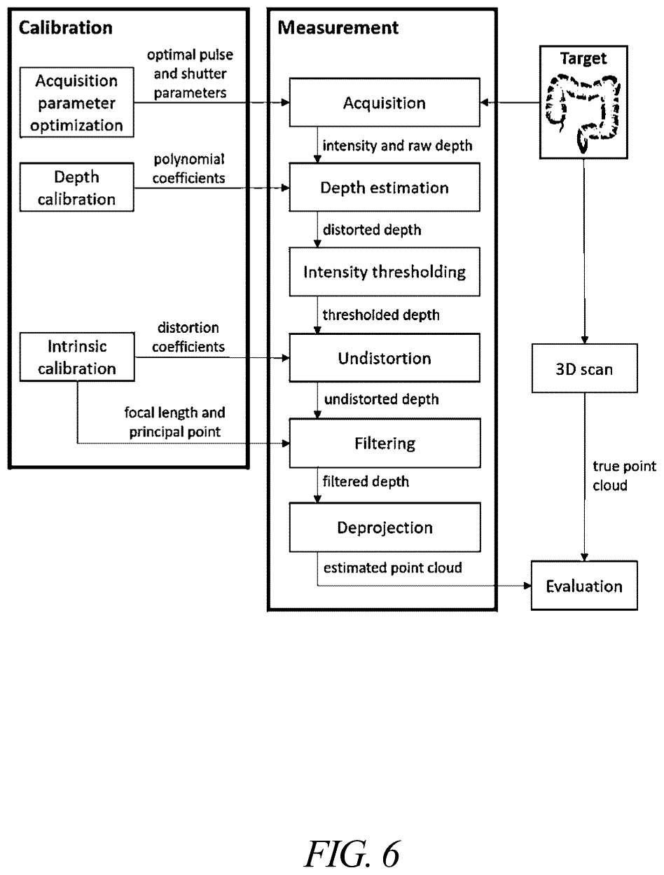

Systems and methods of the present disclosure proved an example pipeline to calibrate a TOF system and to estimate a point cloud of a surgical target. The various operations of an example process are illustrated in FIG. 6.

Acquisition parameter optimization: FIG. 7 shows six different acquisition parameters, where n acquisition cycles per frame comprises a single pulse of width T_{P} transmitted at t=0 followed by three shutters of width T_{S} and hardware gain g including two signal shutters at t=t₀ and t=t₁ and noise compensation shutter at t=t₂ (noise shown as a dark grey horizontal band across the pulse width, e.g., in shutter 2). Based on the minimum (earliest received (Rx) pulse) and maximum (latest Rx pulse), optical path lengths dₘᵢₙ and dₘₐₓ and speed of light c; tₘᵢₙ and tₘₐₓ were computed, which correspond respectively to the earliest and latest times for receiving the pulse rising edge post transmission rising edge. This is shown schematically in FIG. 7, which includes bands in light and medium grey in which the earliest and latest received pulse overlap with Shutter 0 and Shutter 1. These were subsequently used to determine appropriate ranges for T_{S}, t₀, and t₁.

The acquisition parameters governing the pulsed TOF control system include, for example: the number of pulses per frame, laser pulse, width, first signal shutter timing, second signal shutter timing, shutter width, and CCD sensor hardware gain. The set of parameters that minimizes the temporal noise on the depth measurement across a specified depth range was identified, whereby the depth range is defined as a working distance measured from the scope tip. For a given set of acquisition parameters, the optimization process involved averaging the pixel-wise temporal mean and standard deviation of raw depth at the minimum (μ̅ₘ, σ̅ₘ) and maximum (μ̅_{M}, σ̅_{M}) working distance for a normal planar target, and then maximizing the objective function

$$r = \frac{{\overline{μ}}_{M} - {\overline{μ}}_{m}}{{\overline{σ}}_{m} {\overline{σ}}_{M}} .$$

Due to the large space of acquisition parameters, it may not be possible to test all parameter permutations. Therefore, the optimization was confined using a few initial assumptions about system performance and preliminary system tests, each leveraging the fact that the measurements to be made lie within a comparatively narrow working range such as those encountered in endoscopic surgery. The assumptions were made and validated using a small set of manual tests are described below. The assumptions include: (1) the number of pulses may often be increased to achieve increased returned; (2) signal power; for a given target, sensor gain may be increased up to image saturation; (3) shorter shutter widths generally improve images because they tend to reduce noise; (4) signal shutter timing may be used to increase the integrated signal power differential within the working distance (assuming an exponential characteristic to pulse rise and fall curves, this involves timing the first shutter such that its closing corresponds to the minimum possible return time of peak pulse power and timing the second shutter such that its opening corresponds to the minimum possible pulse falling edge return time); and (5) pulse width may be equal to the optical rise time, which may allow peak optical power to be achieved (any longer pulse width may exacerbate heating issues, which may be an error source in TOF systems, and add a flat region to the returned optical power which may add no extra signal power differential across different pulse return times).

Assumptions 1 and 2 were validated by independently varying CCD gain and number of pulses respectively while holding all other parameters constant and observing no significant change in r across a range of values. Assumption 3 was validated by observing an optimum in r for a certain shutter width while holding other parameters constant. Assumptions 4 and 5 were validated first using a theoretical model on a handful of selected acquisition parameter sets, in which received pulses with different widths and exponential rise and fall times, coming from across the distance range, were simulated.

Following these initial checks, an automated optimization was performed on both shutter timings and shutter width, while clamping pulse width, CCD gain, and number of pulses. For shutter timing, ranges were set centering on the theoretically expected optimal positions predicted by Assumption 4, based on the rise time of the 850 nm VCSEL, the pulse width, and total minimum and maximum optical path lengths within the working distance. For shutter width, a range was set centering on the approximated optimal region identified in the initial checks. Clamped pulse width was determined empirically as the minimal value at which the relationship between commanded pulse width and measured optical power became linear. CCD gain was set to the minimum of the range in which r was determined not to be affected. In some cases, a number of pulses was increased.

### Depth Calibration:

Overview: Systems and methods of the present disclosure may provide developing a function to transform the raw depth \hat{d} and intensity p measurements obtained by the system at an selection or set of acquisition parameters to a true or sufficiently true distance from the scope D. The depth calibration workflow develop a function which establish a distance. The method may address one or more of the following:
a. Obtain a depth in physical units.
b. Compute depth from the image plane at the scope tip rather than the tip itself
c. Compensate for depth distortion introduced by the endoscopic fiber optic transmission system, which introduces a field-of-view (FOV) dependent delay to light rays emitted from the endoscope tip due to longer path lengths encountered at higher entry angles.
d. Diminish spatial and temporal noise caused by variable intensity.
e. Diminish variability due to discrepancies between TOF systems.

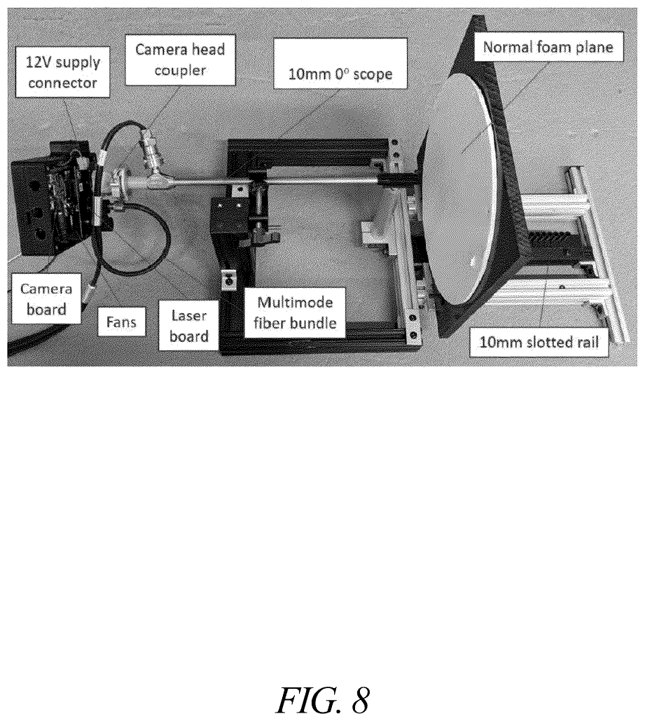

Data collection: As shown in FIG. 8, an example depth calibration setup may comprise an immobilized TOF system and endoscope positioned normally to a plane made of white foam that is movable in one axis. White foam was selected because it is highly reflective in the IR range and thus provided sufficient data for calibrating pixels at higher FOV angles. Along the axis is a series of slots allowing the plane to be immobilized at 10 mm increments. Raw depth and intensity maps were acquired while setting the plane at every slot within a specified working range from the scope tip and simultaneously cycling the laser pulse count (effectively changing the illumination intensity). Sufficient data was collected at every plane position, by acquiring at least ten frames for a given scope distance and pulse count combination.

Analysis: A few different bivariate polynomial fits D(\hat{d}, p)=Σ_{i∈[0,n],j∈[0,n]}a_{i,j}\hat{d}ⁱP^{j} were tested, where n was one of several investigated polynomial orders and a_{i,j} the corresponding coefficients. In order to improve calibration accuracy without additional computational complexity, a unique polynomial model was developed for each pixel, producing a set of VGA parameter arrays of size (n+1)². In each case, model parameters at a pixel were optimized using a least squares approach on all of the sampled raw depth and intensity values, disregarding samples with either a low intensity or undefined depth due to saturated intensity. As a measure of the accuracy of the resulting depth map, the optimized parameter values in each polynomial were used to compute mean and standard deviation of both temporal and spatial error at a selected set of distances and illumination intensities. Temporal statistics were computed on every pixel over ten frames at a given distance, illumination intensity, and polynomial model. Spatial statistics were computed as the mean and standard deviation across pixels in a single image. Acquisition parameters were selected based on the minimum sum of temporal and spatial standard deviation

### Intrinsic Calibration:

Overview: In some cases, systems and methods of the present disclosure may comprise computing the distortion coefficients \vec{d}, focal lengths fₓ and f_{y} and principal points cₓ and c_{y} of each scope assembly. The distortion coefficients may be useful to digitally eliminate barrel distortions due to lens aberrations, while the focal length and principal point may be useful to generate a point cloud, the details of which are described in the following section.

Data collection: A 200-frame video of a moving 9×6 checkerboard (side length 9.9 mm) manually positioned at various locations and orientations in the scene was recorded. The checkerboard was well illuminated at all times and moved slowly to prevent blurring effects.

Analysis: Using OpenCV's checkerboard detection algorithm, the subset of frames (in which an entire checkerboard was detected) was selected. Next, the frames were randomly shuffled, divided into groups of 30, and a camera calibration was performed on each group. This process was repeated 5 times and then a mean and standard deviation of all intrinsic parameters from all iterations was computed. The repetition and standard deviation were useful to determine the consistency of the results and mitigate any statistical noise due to the specific set of selected images.

### Evaluation of 3D Measurement

In some cases, systems and methods of the present disclosure may comprise evaluating a quality of the 3D measurement. Point clouds on selected targets were computed using a combination of the depth and intrinsic calibration parameters. The point clouds were evaluated using ground truth obtained from a commercial 3D scanner.

Point cloud evaluation: Point cloud computation and evaluation comprises the following steps, also summarized in FIG. 6: Depth estimation (computation of a distorted pixel-wise depth map using acquired intensity and raw depth frames); Intensity thresholding (masking of depth values for which the received intensity was outside of a specified range); Distortion compensation (undistortion of the pixel-wise depth map using \vec{d} to produce undistorted pixel-wise depth map); Filtering (application of a spatial and temporal filter to the depth map); De-projection (estimation of a point cloud using filtered depth map Z and intrinsic parameters, which involves computing coordinates within the imaging plane X_{u,v} and Y_{u,v} for each pixel u, v as X_{u,v}=Z_{u,v}(u−c_{X})/f_{X} and Y_{u,v}=Z_{u,v}(v−c_{y})/f_{y}); and Evaluation (registration of the computed point cloud to a reference point cloud and computation of nearest neighbor errors).

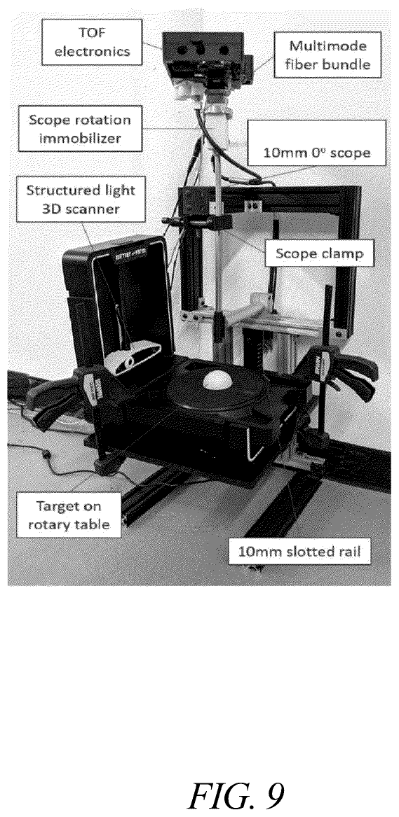

Scanner evaluation: To evaluate the accuracy of the endoscopic TOF system, a commercial structured-light scanner was used for ground truth. The performance of the scanner was first evaluated on a 3D printed target with a radius. FIG. 9 shows an example setup for point cloud evaluation. Error was computed as the mean distance between all points in the measured cloud to their nearest neighbors in the aligned reference cloud. In this case, the reference point cloud was densely sampled from a model of a perfect hemisphere, while the measured point cloud was obtained using the 3D scanner. Point clouds were aligned using manual approximation followed by the iterative closest point algorithm. The Open3D library was used for all computations related to point clouds.

Endoscopic TOF system evaluation in ex vivo tissue: The endoscopic TOF system was evaluated by its ability to produce an accurate point cloud on a biological target as determined by ground truth obtained from the scanner. The selected target was a porcine kidney, chosen for its variable surface geometry and relatively high rigidity (thus minimizing the deformation that occurs during a rotary scan). The setup for this data collection is visualized in FIG. 9. Point clouds were computed at a scope-tip-to-target distance of 130 mm, with maximum TOF illumination power. All frames were taken through the pipeline described in FIG. 6, with point clouds being computed for each of a selected set of temporal and spatial filter parameters within a region of interest manually selected to contain the kidney target. Error in each case was computed using the same method described in section II-D.2.

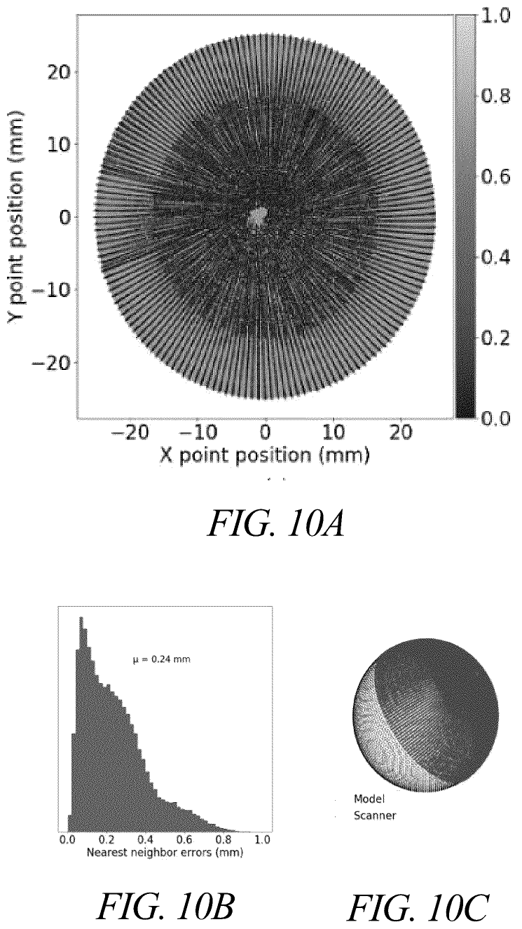

Results from 3D scanner evaluation are shown in FIGS. 10A-10C. FIG. 10A shows an error map projected onto flat side of hemisphere and overlaid on the modelled hemisphere area while FIG. 10B shows an error histogram directly attained from error map in FIG. 10A. FIG. 10C comprises a rendering of registered 3D point clouds from 3D scanner and down-sampled hemisphere model.

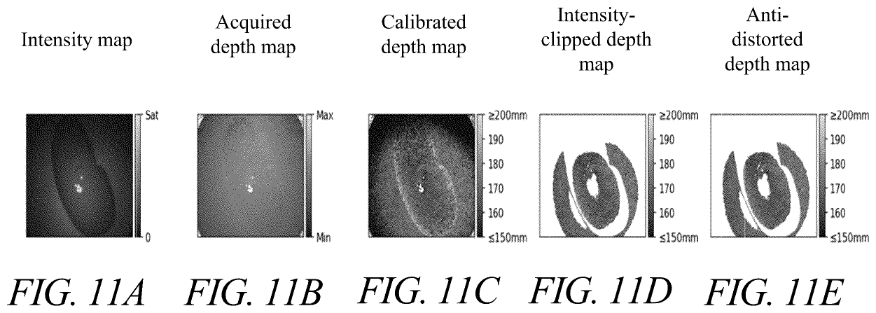

Results from acquisition and preprocessing of a depth map from a representative frame are shown in FIGS. 11A-11E. FIGS. 11A-11E respectively show acquired intensity map, acquired depth map, calibrated depth map, intensity-clipped depth map, and anti-distorted depth map.

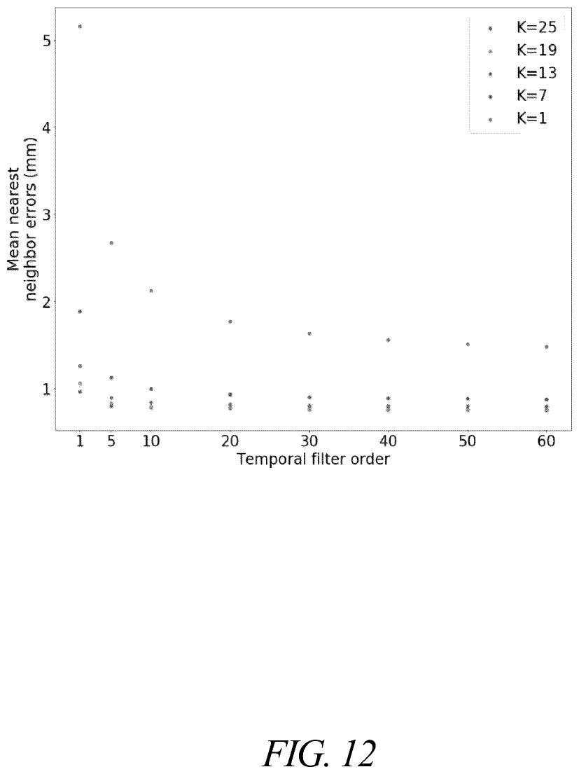

FIG. 12 shows mean nearest neighbor errors from performing a sweep of spatial and temporal filtering parameters.

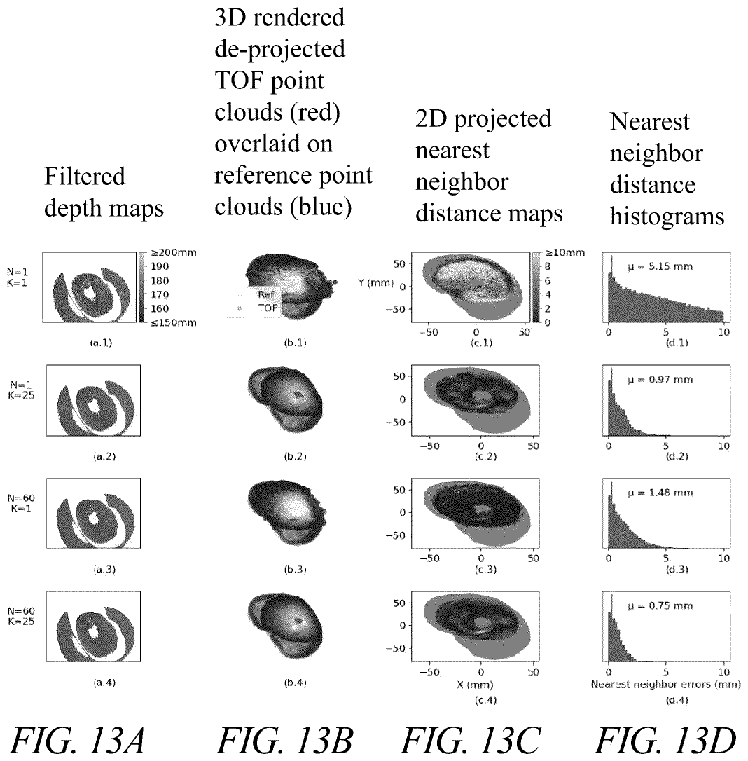

Evaluation of a few point clouds from selected sets of filtering parameters are shown in FIGS. 13A-D. The four panels of FIG. 13A, FIG. 13B, FIG. 13C, and FIG. 13D respectively show filtered depth maps, 3D rendered de-projected TOF point clouds overlaid on reference point clouds, 2D projected nearest neighbor distance maps, and nearest neighbor distance histograms. Each row x corresponds to a different set of filter parameters from those swept in FIG. 12.

### Scanner Evaluation

A rigid 3D printed hemisphere having a diameter was used to evaluate the performance of the 3D scanner used in the study. FIG. 10A demonstrates that most scanned points are accurate to within 0.2 mm, a value which is reasonably close to that reported by the manufacturer (0.1 mm). This value may have additionally been affected by the layer thickness of the 3D printer (0.1 mm). For the purpose of this study, a0.2 mm was considered acceptable as ground truth accuracy given that this is likely within the range of the soft organ deformation that might occur during a scan, whether due to rotation or fluid seepage that can occur over the time period of a scan.

### TOF System Evaluation

Depth and intensity acquisition: The results from this evaluation are shown in FIG. 11A-11E. FIG. 11A shows several saturated regions where specular reflection occurs in the scene, along with regions of nearly no received intensity in the corners. Correspondingly in FIG. 11B, the same regions containing undefined depth values. This feature may be an artifact of the TOF measurement system, which may not be able to distinguish variability in pulse arrival time with sensor saturation or low received signal. The “donut-shaped” pattern of this illumination, which includes both specular reflection in the middle of the image and vignetting at the edges, is largely a function of the transmit optics, which directly couple a diffused VCSEL to a multi-mode fiber bundle of limited numerical aperture. This likely produces a Gaussian-like illumination profile of angular width smaller than the field of view of the imaging system, resulting in the vignetting effect. The bivariate polynomial calibration model developed in the study then takes as input FIG. 11A and FIG. 11B and produces the calibrated depth map in FIG. 11C, which is undefined in the same regions as the raw depth.

Depth map pre-processing: The calibrated depth map features outlier values around the area of specular reflection. Therefore, the intensity threshold was manually selected for pixels considered in the analysis, removing pixels whose received power went above this threshold. The result is in FIG. 11D, in which the undefined region has expanded with the additional removal. Besides having the effect of reducing outliers, this procedure also prevented affecting even more pixels with these outlier values during anti-distortion, which relies on weighted averaging of nearest neighbors. FIG. 11E shows the results of distortion compensation, which produce minimal modification of the image due to minimal distortion from the 10 mm laparoscope used in the study.

Point cloud evaluation: In the next step of the analysis, the depth map was filtered using a set of pre-selected spatial and temporal filter orders and evaluated the results of the corresponding de-projected point cloud. Across both spatial and temporal orders, nearest neighbor error decreases asymptotically toward an optimum of approximately 0.75 mm. Across temporal filter orders, most of the benefits are encountered up to an order of 30, which corresponds to 1 second for the 30 Hz TOF processor. Across spatial filter orders, most of the improvement is seen up to a kernel size of 19. The lowest nearest neighbor error is attained using a combination of high spatial and temporal filter orders, indicating the presence of both temporal and spatial noise in the signal. Notably, a sub-millimeter error may be attained using a spatial filter, indicating that it can be attained in a single-shot provided that the imaged target is smooth enough. Also, it is clear from the data that both temporal and spatial noise are present in the original point clouds, such that both domains of filtering may be useful to produce the optimal result.

TOF point cloud errors can be contextualized by comparing them to the resolution of state-of-the-art medical 3D imaging modalities. Two of the most widespread imaging modalities are computed tomography (CT) and magnetic resonance imaging (MRI). The former attains spatial and temporal resolution in the range of 0.5 to 0.625 mm and 66 to 210 milliseconds (ms), respectively, while for the latter these values are 8 to 10 mm and 20 to 55 ms, depending on the application. The TOF filter parameter results presented in FIG. 12 can be viewed in a parallel context, in which the number of frames combined with knowledge of the system frame rate can be used as a surrogate for temporal resolution and the nearest neighbor error can be used as a surrogate for spatial resolution. As FIG. 12 shows, laparoscopic TOF can outperform MRI, and falls just short of CT but at a comparable temporal resolution (2 to 7 frames at 30 Hz). Of course, the TOF measurement can be attained real-time and continuously throughout a procedure, using a handheld laparoscopy setup, thus promising several potential future applications around measurement not enabled by MRI or CT. Used alone, sub-millimeter laparoscopic TOF may eliminate the need for a pre-operative CT or MRI in certain cases where targets have variable geometry (such as a cyst or rapidly growing tumor), are difficult to detect using state-of-the-art scanning approaches (such as a hernia), or are not present during the time of the scan (such as an anastomosis). Used in combination with MRI or CT, TOF may be used for real-time registration of tissue geometry to the pre-operative scan, thus allowing more accurate localization of otherwise invisible underlying structures.

The systems and methods described herein can provide for greater accuracy across a variety of tissue targets, scopes, and/or distances. In some instances, a real-time measurement application can be developed or integrated with any of the systems and methods described herein. In another aspect, the combination of the TOF system with an aligned RGB video may be used to both increase 3D measurement accuracy using color compensation and also texture the tissue surface geometry for better 3D perception.

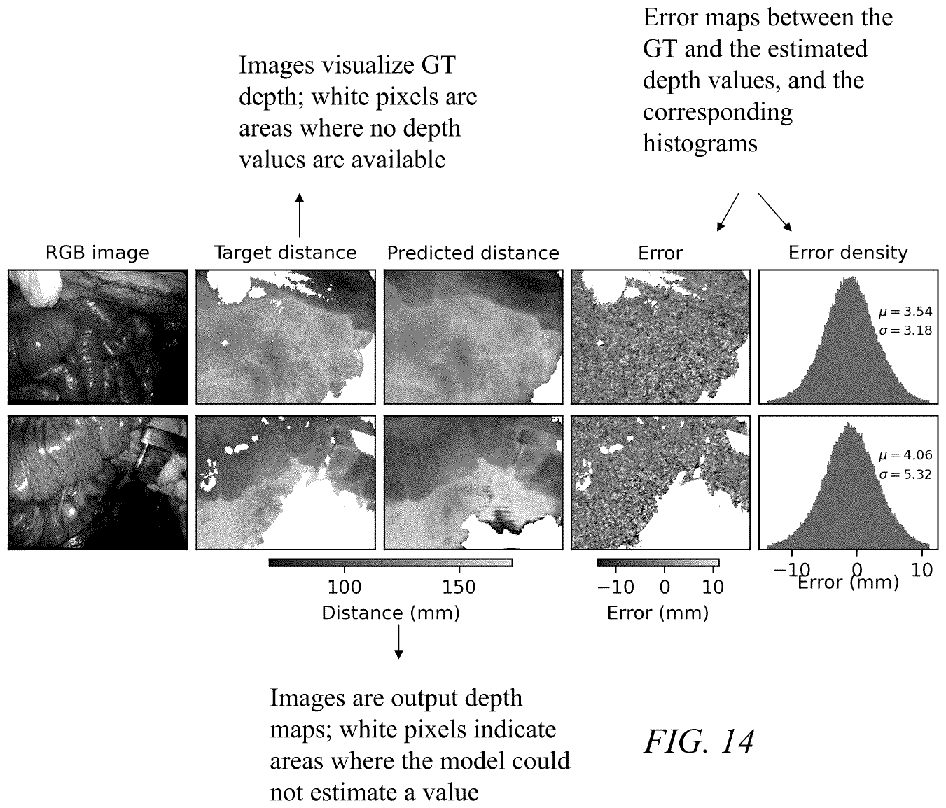

In FIG. 14, a FastDepth model is employed with a MobileNet backbone, to target real-time deployment in order to estimate depth. The model can be trained and tested on a dataset acquired in a model (e.g., a porcine animal model). The dataset may comprise an RGB dataset, used as model input, and an aligned depth stream used as ground truth (GT). The GT can be measured with sub-millimeter accuracy using a monocular time of flight (TOF) laparoscopic system. Any pixels for which a depth value is unavailable or unreliable due to saturation or low signal can be masked in both streams and not used in training. The model can be trained for fifty epochs using a smooth L1 loss. To assess model performance, the percentage of pixels with values within 25% of the GT were measured.

Two representative examples are shown in FIG. 14. The left two columns show the RGB images and corresponding depth GT. The white pixels are areas where no depth values are available in the GT image. Column three shows the output depth maps from the trained model. The white pixels indicate areas where the model did not estimate a value. The error maps between the GT and the estimated depth values and the corresponding histograms are shown in the two right most columns. The average errors for the two examples value were 3.54±3.18 mm and 4.06±5.32 mm. For the entire validation dataset, the percentage of pixels within 25% of the expected value was 71.8%.

The present disclosure provides a surgical depth mapping method compatible with standard laparoscopes and surgical workflow. Mean absolute error from two RGB scenes suggests utility for real-time clinical applications such as normalization of fluorescence by distance (e.g., allowing quantification of perfusion using fluorescent dyes). In some embodiments, the model makes reasonable predictions where GT is absent or unreliable. This is most evident in its robustness on surgical tools (e.g., gauze and metal retractor) and specular highlights. Additionally, the model may be configured to produce less noisy output than the GT due to internal smoothing. In some cases, improved GT can be used to reduce model error. Methods and systems of the present disclosure can be implemented by way of one or more algorithms. An algorithm can be implemented by way of software upon execution by the central processing unit 2005. For example, the algorithm may be configured to generate one or more image overlays based on the one or more medical images generated using at least a portion of the light signals reflected from the surgical scene. The one or more image overlays may comprise, for example, TOF imaging data, laser speckle imaging data, fluorescence imaging data, and/or RGB imaging data associated with the surgical scene or one or more anatomical features or physiological characteristics of the surgical scene.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A system for medical imaging, comprising:
an imaging sensor configured to receive a plurality of light signals reflected from an illuminated surgical scene and having,
a first imaging unit configured for time of flight (TOF) imaging;
a second imaging unit configured for laser speckle imaging and
an optical element configured to (i) direct a first set of light signals to the first imaging unit and (ii) direct a second set of light signals to the second imaging unit; and
an image processing module operatively coupled to the first imaging unit and the second imaging unit, wherein the image processing module is configured to generate one or more images of monocular laparoscopic depth estimation of the surgical scene based on (i) the first set of light signals and (ii) the second set of light signals reflected from one or more features or portions of the surgical scene.

2. (canceled)

3. (canceled)

4. (canceled)

5. (canceled)

6. (canceled)

7. The system of claim 1, wherein the optical element is further configured to (iii) direct a third set of light signals to a third imaging unit configured for Red, Green, Blue RGB imaging.

8. (canceled)

9. (canceled)

10. (canceled)

11. (canceled)

12. (canceled)

13. (canceled)

14. The system of claim 1, wherein the image processing module is configured to generate one or more images for visualizing fluorescence in the surgical scene, based on one or more light signals received at the first imaging unit.

15. The system of claim 1, wherein the image processing module is configured to utilize image interpolation to account for a plurality of different frame rates and exposure times associated with the first and second imaging units when generating the one or more images of the surgical scene.

16. The system of claim 1, wherein the image processing module is configured to quantify or visualize perfusion of a biological fluid in, near, or through the surgical scene based on the one or more images of the surgical scene.

17. The system of claim 1, wherein the image processing module is configured to generate one or more perfusion maps for one or more biological fluids in or near the surgical scene, based on the one or more images of the surgical scene.

18. The system of claim 17, wherein the image processing module is configured to update, refine, or normalize the one or more perfusion maps based on a distance between (i) a scope through which the plurality of light signals is transmitted and (ii) one or more pixels of the one or more images.

19. The system of claim 17, wherein the image processing module is configured to update, refine, or normalize the one or more perfusion maps based on a position, an orientation, or a pose of a scope through which the plurality of light signals is transmitted relative to one or more pixels of the one or more images.

20. The system of claim 17, wherein the image processing sensor comprises a depth sensor configured to update, refine, or normalize the one or more perfusion maps based on depth information or a depth map associated with the surgical scene, wherein the depth information or the depth map is derived from or generating using the first set of light signals.

21. The system of claim 20, wherein the image processing module is configured to determine a pose of a scope through which the plurality of light signals is transmitted relative to one or more pixels of the one or more images, based on the depth information or the depth map.

22. The system of claim 21, wherein the image processing module is configured to update, refine, or normalize one or more velocity signals associated with the perfusion map based on the pose of the scope relative to the surgical scene.

23. The system of claim 17, wherein the image processing module is configured to update, refine, or normalize the one or more perfusion maps based on a type of tissue detected or identified within the surgical scene.

24. The system of claim 17, wherein the image processing module is configured to update, refine, or normalize the one or more perfusion maps based on an intensity of at least one of the first and second set of light signals, wherein the intensity is a function of a distance between a scope through which the plurality of light signals is transmitted and one or more pixels in the surgical scene.

25. The system of claim 17, wherein the image processing module is configured to update, refine, or normalize the one or more perfusion maps based on a spatial variation of an intensity of at least one of the first and second set of light signals across the surgical scene.

26. The system of claim 1, wherein the image processing module is configured to infer a tissue type based on an intensity of one or more light signals reflected from the surgical scene, wherein the one or more reflected light signals comprise at least one of the first set of light signals and the second set of light signals.

27. (canceled)

28. (canceled)

29. (canceled)

30. (canceled)

31. (canceled)

32. (canceled)

33. (canceled)

34. (canceled)

35. (canceled)

36. The system of claim 1, wherein the image processing module is configured to use at least one of the first set of light signals and the second set of light signals to determine a motion of a scope, a tool, or an instrument relative to the surgical scene.

37. The system of claim 7, wherein the image processing module is configured to (i) generate one or more depth maps or distance maps based on the first set of light signals or the second set of light signals, and (ii) use the one or more depth maps or distances map to generate one or more machine-learning based inferences, which one or more machine-learning based inferences comprise at least one of automatic video de-identification, image segmentation, automatic labeling of tissues or instruments in or near the surgical scene, and optimization of image data variability based on one or more normalized RGB or perfusion features.

38. The system of claim 1, wherein the image processing module is configured to (i) generate one or more depth maps or distance maps based on at least one of the first set of light signals and the second set of light signals, and (ii) use the one or more depth maps or distances map to perform temporal tracking of perfusion or to implement speckle motion compensation.

39. (canceled)

40. (canceled)

41. The system of claim 1, further comprising a calibration module configured to perform depth calibration on one or more depth maps generated using the image processing module.

42. The system of claim 41, wherein depth calibration comprises updating the one or more depth maps by sampling multiple targets at one of (i) multiple distances or (ii) multiple illumination intensities.

43. (canceled)

44. (canceled)

45. (canceled)

46. (canceled)

47. (canceled)

48. (canceled)

49. (canceled)

50. (canceled)
